(19) United States
(12) Patent Application Publication
(10) Pub. No.: US 2016/0183854 A1
(43) Pub. Date: Jun. 30, 2016

(54) ANALYTE MONITORING SYSTEMS, DEVICES, AND METHODS

(71) Applicant: ABBOTT DIABETES CARE INC., Alameda, CA (US)

(72) Inventor: Tony S. Lee, Alameda, CA (US)

(21) Appl. No.: 14/951,193

(22) Filed: Nov. 24, 2015

Related U.S. Application Data
(60) Provisional application No. 62/084,514, filed on Nov. 25, 2014.
(60) Provisional application No. 62/161,776, filed on May 14, 2015.
(60) Provisional application No. 62/161,764, filed on May 14, 2015.

(51) Int. Cl. A61B 5/1473; A61B 5/145; A61B 5/00

(52) U.S. Cl. CPC A61B 5/1473; A61B 5/6832; A61B 5/14532

(57) ABSTRACT

An analyte monitoring device includes an electric power source, an analyte sensor, and sensor electronics. The analyte sensor includes a plurality of electrodes, including an in vivo portion of the analyte sensor configured for fluid contact with a bodily fluid under a skin layer. The sensor electronics includes a data processing section configured to process one or more signals received from the analyte sensor.

### CROSS-REFERENCE TO RELATED APPLICATIONS

This application claims priority to U.S. Provisional Patent Application No. 62/084,514 filed Nov. 25, 2014 entitled Analyte Monitoring Devices and Systems. This application also claims priority to U.S. Provisional Patent Application No. 62/161,776 filed May 14, 2015 entitled Analyte Monitoring Devices and Systems. This application also claims priority to U.S. Provisional Patent Application No. 62/161,764 filed May 14, 2015 entitled One-Time Electronic Switch. The contents of all of these patent applications are incorporated by reference herein in their entirety and for all purposes.

### BACKGROUND

The association of chronic hyperglycemia and the devastating long-term complications of diabetes was clearly established by the Diabetes Control and Complication Trial (DCCT) (The Diabetes Control and Complications Trial Research Group. “The effect of intensive treatment of diabetes on the development and progression of long-term complications of insulin-dependent diabetes mellitus” N Engl J Med 329: 978-986, 1993; Santiago J V “Lessons from the Diabetes Control and Complications Trial” Diabetes 1993, 42: 1549-1554).

The DCCT found that in patients receiving intensive insulin therapy, there was a reduced risk of 76% for diabetic retinopathy, 50% for diabetic nephropathy and 60% for diabetic neuropathy. The long-term benefits of tight glycemic control have been further substantiated by the Epidemiology of Diabetes Interventions and Complications study which found over a 50% reduced risk of macrovascular disease as a result of intensive insulin therapy (The Diabetes Control and Complications Trial/Epidemiology of Diabetes Intervention and Complication (DCCT/EDIC) Study Group, “Intensive diabetes treatment and cardiovascular disease in patients with type 1 diabetes”, 353, 2643-2653, 2005).

However, the DCCT found that patients receiving intensive insulin therapy were at a threefold increased risk of severe hypoglycemia. Patients adhering to intensive insulin therapy regimens were found to have lowered thresholds for activation of neurogenic warning systems and consequently were at increased risk for more severe hypoglycemic events. (Amiel S A, Tamborlane W V, Simonson D C, Sherwin R S., “Defective glucose counterregulation after strict glycemic control of insulin-dependent diabetes mellitus.” N Engl J. Med. 1987 28; 316(22):1376-83).

The increased risk of hypoglycemia and the fear associated with patients' perception of that risk has been cited as the leading obstacle for patients to achieve the targeted glycemic levels (Cryer P E. “Hypoglycaemia: The limiting factor in the glycemic management of type I and type II diabetes” Diabetologia, 2002, 45: 937-948). In addition to the problem of chronic hyperglycemia contributing to long-term complications and the problem of acute iatrogenic hypoglycemia contributing to short-term complications, recent research suggests that transient episodes of hyperglycemia can lead to a wide range of serious medical problems besides previously identified microvascular complications as well as macrovascular complications such as increased risk for heart disease. (Haffner S “The importance of postprandial hyperglycemia in development of cardiovascular disease in people with diabetes” International Journal of Clinical Practice, 2001, Supplement 123: 24-26; Hanefeld M: “Postprandial hyperglycemia: noxious effects on the vessel wall” International Journal of Clinical Practice, 2002, Supplement 129: 45-50).

Additional research has found that glycemic variation and the associated oxidative stress may be implicated in the pathogenesis of diabetic complications (Hirsh I B, Brownlee M “Should minimal blood glucose variability become the gold standard of glycemic control?” J of Diabetes and Its Complications, 2005, 19: 178-181; Monnier, L., Mas, E., Ginet, C., Michel, F., Villon L, Cristol J-P, and Collette C, “Activation of oxidative stress by acute glucose fluctuations compared with sustained chronic hyperglycemia in patients with type 2 diabetes”. JAMA 2006, 295, 1681-1687). Glycemic variation has also been identified as a possible explanation for the increased prevalence of depression in both type 1 and type 2 diabetes (Van der Does F E. De Neeling J N, Snoek F J, Kostense P J, Grootenhuis P A, Bouter L M, and R J Heine: Symptoms and well-being in relation to glycemic control in type II diabetes Diabetes Care, 1996, 19: 204-210; De Sonnaville J J. Snoek F J. Colly L P. Deville W. Wijkel D. Heine R J: “Well-being and symptoms in relation to insulin therapy in type 2 diabetes” Diabetes Care, 1998, 21:919-24; Cox D J, Gonder-Frederick L A, McCall A, et al. “The effects of glucose fluctuation on cognitive function and QOL: the functional costs of hypoglycaemia and hyperglycaemia among adults with type 1 or type 2 diabetes” International Journal of Clinical Practice, 2002, Supplement 129: 20-26).

The potential benefits of in vivo glucose monitoring (e.g., continuous glucose monitoring and flash glucose monitoring) have been recognized by numerous researchers in the field (Skyler J S “The economic burden of diabetes and the benefits of improved glycemic control: the potential role of a continuous glucose monitoring system” Diabetes Technol Ther 2 (Suppl 1): S7-S12, 2000; Tansey M J, Beck R W, Buckingham B A, Mauras N, Fiallo-Scharer R, Xing D, Kollman C, Tamborlane W V, Ruedy K J, “Accuracy of the modified Continuous Glucose Monitoring System (CGMS) sensor in an outpatient setting: results from a diabetes research in children network (DirecNet) study.” Diab. Tech. Ther. 7(1):109-14, 2005; Klonoff, D C: “Continuous glucose monitoring: Roadmap for 21st century diabetes therapy” Diabetes Care, 2005, 28: 1231:1239). Accurate and reliable real-time in vivo glucose monitoring devices have the ability to alert patients of high or low blood sugars that might otherwise be undetected by episodic capillary blood glucose measurements.

In vivo glucose monitors have the potential to permit more successful adherence to intensive insulin therapy regimens and also to enable patients to reduce the frequency and extent of glycemic fluctuations. However, the development of this technology has proceeded more slowly than anticipated. For example, two recent comprehensive reviews of decades of research in the field cited the lack of accuracy and reliability as the major factor limiting the acceptance of this new technology as well as the development of an artificial pancreas (Chia, C. W. and Saudek, C. D., “Glucose sensors: toward closed loop insulin delivery” Endocrinol. Metab. Clin. N. Am., 33, 174-195, 2004; Hovorka, R. “Continuous glucose monitoring and closed-loop systems” Diabet. Med. 23, 1-12, 2006).

As in vivo analyte monitoring becomes more prevalent, of use are in vivo analyte sensors and systems that are accurate to such a high degree that confirmatory analyte measurement are not needed to verify the in vivo sensing measurements, e.g., prior to a user relying on the in vivo measurements. Also of interest are such sensors that work in concert with a drug delivery device.

The detection and/or monitoring of analyte levels, such as glucose, ketones, lactate, oxygen, hemoglobin A1C, or the like, can be vitally important to the health of an individual having diabetes. Diabetics generally monitor their glucose levels to ensure that they are being maintained within a clinically safe range, and may also use this information to determine if and/or when insulin is needed to reduce glucose levels in their bodies or when additional glucose is needed to raise the level of glucose in their bodies.

Growing clinical data demonstrates a strong correlation between the frequency of glucose monitoring and glycemic control. Despite such correlation, many individuals diagnosed with a diabetic condition do not monitor their glucose levels as frequently as they should due to a combination of factors including convenience, testing discretion, pain associated with glucose testing, and cost. Thus, needs exist for improved in vivo glucose monitoring systems, devices, and methods.

### SUMMARY

Generally, the present disclosure relates to systems, devices, and methods for the monitoring of the level of an analyte using an in vivo sensor. Embodiments include sensors in which at least a portion of the sensor is adapted to be positioned beneath a skin surface of a user.

In certain embodiments, improved applicator devices and methods are described that enable insertion of the sharp and sensor into the user's body with a dampening mechanism to absorb extraneous forces applied to the sharp and sensor. The dampening mechanism can reduce the likelihood of insertion at an improper angle, and thereby reduce the likelihood of poor placement of the in vivo sensor.

Also provided in certain embodiments are devices and methods that enable mechanical and electrical activation of an in vivo sensor. These embodiments enable a sensor control device to be shipped and stored in a low power state, and enable the user to mechanically activate the sensor control device such that it transitions from a low power state to a relatively higher power state for use in monitoring the user's analyte level.

Also provided are certain embodiments of analyte monitoring systems that are adapted for providing clinically accurate analyte data, i.e., data with accuracy sufficient so that a user may confidently rely on the sensor results, e.g., to manage a disease condition and/or make a healthcare decision based thereon. Accordingly, sensors capable of providing clinically accurate (and clinically relevant) analyte information to a user are provided.

Embodiments include in vivo analyte monitoring systems that do not require additional analyte information obtained by a second system and/or sensor to confirm the results reported by the analyte monitoring system.

Embodiments also include high accuracy in vivo analyte sensors and systems with drug delivery systems e.g., insulin pumps, or the like. A communication link (e.g., by cable or wirelessly such as by infrared (IR) or RF link or the like) may be provided for transfer of data from the sensor to the drug delivery device. The drug delivery device may include a processor to determine the amount of drug to be delivered using sensor data, and may deliver such drug automatically or after user direction to do so.

Also provided are highly accurate in vivo analyte sensors and methods of analyte monitoring using the same.

These and other objects, features and advantages of the present disclosure will become more fully apparent from the following detailed description of the embodiments, the appended claims and the accompanying drawings.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a high level diagram depicting an example embodiment of an analyte monitoring system for real time analyte (e.g., glucose) measurement, data acquisition and/or processing.

FIG. 2A is a block diagram depicting an example embodiment of a reader device configured as a smartphone.

FIG. 2B is a block diagram depicting an example embodiment of a sensor control device.

FIGS. 3A-F depict steps in an example embodiment of a process of assembling a sensor control device and delivering that device to the user's body.

FIGS. 3G-K are cross-sectional views depicting an example embodiment of an applicator device during delivery of a sensor control device.

FIGS. 4A-D are various views depicting an example embodiment of a sensor control device.

FIG. 5A is a perspective view of an example embodiment of a sharp and sensor module.

FIG. 5B is a perspective view of an example embodiment of a sharp module.

FIG. 5C is a perspective and partially exploded view of an example embodiment of a sharp and sensor module.

FIG. 5D is a perspective view depicting an example embodiment of an elastic dampener.

FIGS. 6A-B are top-down perspective views depicting example embodiments of a switch for activating a sensor control device.

FIG. 7 is a schematic depicting an example embodiment of switch circuity for activating a sensor control device.

FIGS. 8-12 illustrate a first test series and sensory attributes using an example embodiment of an analyte monitoring system.

FIG. 8 illustrates a profile of glucose response over 14 days of sensor wear in accordance with the first test series.

FIG. 9 illustrates Consensus Error Grid Analysis comparing the analyte monitoring system readings with capillary blood glucose reference using the blood glucose meter built into the reader of the system in accordance with the first test series.

FIG. 10 is a histogram of Mean Absolute Relative Difference (MARD) per sensor in accordance with the first test series.

FIG. 11 illustrates stability of accuracy across 14 days of sensor wear with the following legend: Green (bottom)=Zone A, Blue (middle or top)=Zone B, Red (top)=Zone C in accordance with the first test series.

FIG. 12 illustrates accuracy of the analyte monitoring system as a function of various factors or patient characteristics. Abbreviation Key: BMI=Body Mass Index; Insulin Ad=Insulin Administration; HbA1c=Hemoglobin A1c.

FIGS. 13A-16 illustrate a second test series and sensory attributes using an example embodiment of an analyte monitoring system.

FIGS. 13A-B illustrate Consensus and Clarke Error Grid Analyses comparing flash glucose monitoring system sensor readings with capillary blood glucose reference using BG meter built into the reader in accordance with the second test series.

FIG. 14 is a histogram of mean absolute relative difference (MARD) per sensor in accordance with the second test series.

FIG. 15 illustrates the stability of accuracy across 14 days of flash glucose monitoring system sensor wear in accordance with the second test series.

FIG. 16 illustrates accuracy of the Flash Glucose Monitoring System sensor as a function of various factors or patient characteristics according to the second test series.

### DETAILED DESCRIPTION

A number of systems and methods have been developed for the automatic monitoring of the analyte(s), like glucose, in bodily fluid such as in the blood stream, in interstitial fluid (“ISF”), dermal fluid of the dermal layer, or in other biological fluid. Some of these systems are configured so that at least a portion of a sensor is positioned below a skin surface of a user, e.g., in a blood vessel or in the subcutaneous tissue of a user, to obtain information about at least one analyte of the body.

As such, these systems can be referred to as “in vivo” monitoring systems. In vivo analyte monitoring systems include “Continuous Analyte Monitoring” systems (or “Continuous Glucose Monitoring” systems) that can broadcast data from a sensor control device to a reader device continuously without prompting, e.g., automatically according to a broadcast schedule. In vivo analyte monitoring systems also include “Flash Analyte Monitoring” systems (or “Flash Glucose Monitoring” systems or simply “Flash” systems) that can transfer data from a sensor control device in response to a scan or request for data by a reader device, such as with an Near Field Communication (NFC) or Radio Frequency Identification (RFID) protocol. In vivo analyte monitoring systems can also operate without the need for finger stick calibration.

The in vivo analyte monitoring systems can be differentiated from “in vitro” systems that contact a biological sample outside of the body (or rather “ex vivo”) and that typically include a meter device that has a port for receiving an analyte test strip carrying bodily fluid of the user, which can be analyzed to determine the user's blood sugar level. While in many of the present embodiments the monitoring is accomplished in vivo, the embodiments disclosed herein can be used with in vivo analyte monitoring systems that incorporate in vitro capability, as well has purely in vitro or ex vivo analyte monitoring systems.

The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “reader device” (or simply a “reader”), “handheld electronics” (or a handheld), a “portable data processing” device or unit, a “data receiver,” a “receiver” device or unit (or simply a receiver), or a “remote” device or unit, to name a few. Other devices such as personal computers have also been utilized with or incorporated into in vivo and in vitro monitoring systems.

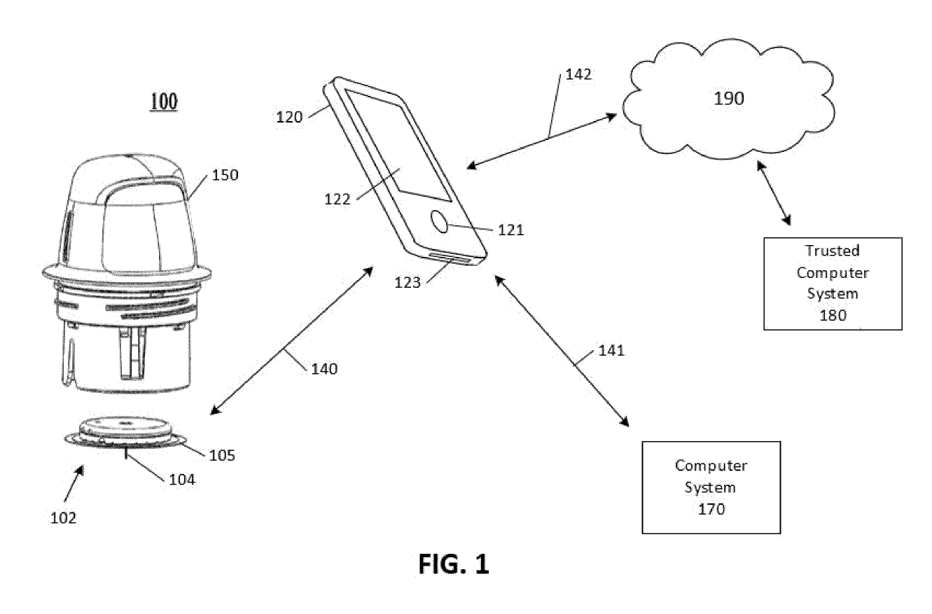

FIG. 1 is an illustrative view depicting an example in vivo analyte monitoring system 100 with which any and/or all of the embodiments described herein can be used. System 100 can have a sensor control device 102 and a reader device 120 that communicate with each other over a local communication path (or link) 140, which can be wired or wireless, and uni-directional or bi-directional. In embodiments where path 140 is wireless, any near field communication (NFC) protocol, RFID protocol, Bluetooth or Bluetooth Low Energy protocol, Wi-Fi protocol, proprietary protocol, or the like can be used, including those communication protocols in existence as of the date of this filing or their later developed variants.

Bluetooth is a well-known standardized short range wireless communication protocol, and Bluetooth Low Energy is a version of the same that requires less power to operate. Bluetooth Low Energy (Bluetooth LE, BTLE, BLE) is also referred to as Bluetooth Smart or Bluetooth Smart Ready. A version of BTLE is described in the Bluetooth Specification, version 4.0, published Jun. 30, 2010, which is explicitly incorporated by reference herein for all purposes. The term “NFC” applies to a number of protocols (or standards) that set forth operating parameters, modulation schemes, coding, transfer speeds, frame format, and command definitions for NFC devices. The following is a non-exhaustive list of examples of these protocols, each of which (along with all of its sub-parts) is incorporated by reference herein in its entirety for all purposes: ECMA-340, ECMA-352, ISO/IEC 14443, ISO/IEC 15693, ISO/IEC 18000-3, ISO/IEC 18092, and ISO/IEC 21481.

Reader device 120 is also capable of wired, wireless, or combined communication with either or both of: a local computer system 170 over communication path (or link) 141 and with a network 190 over communication path (or link) 142. Reader device 120 can communicate with any number of entities through network 190, which can be part of a telecommunications network, such as a Wi-Fi network, a local area network (LAN), a wide area network (WAN), the internet, or other data network for uni-directional or bi-directional communication. A trusted computer system 180 can be accessed through network 190. In an alternative embodiment, communication paths 141 and 142 can be the same path. All communications over paths 140, 141, and 142 can be encrypted and sensor control device 102, reader device 120, remote computer system 170, and trusted computer system 180 can each be configured to encrypt and decrypt those communications sent and received.

Variants of devices 102 and 120, as well as other components of an in vivo-based analyte monitoring system that are suitable for use with the system, device, and method embodiments set forth herein, are described in US Patent Application Publ. No. 2011/0213225 (the '225 Publication), which is incorporated by reference herein in its entirety for all purposes.

Sensor control device 102 can include a housing 103 containing in vivo analyte monitoring circuitry and a power source. The in vivo analyte monitoring circuitry can be electrically coupled with an analyte sensor 104 that can extend through an adhesive patch 105 and project away from housing 103. Adhesive patch 105 contains an adhesive layer (not shown) for attachment to a skin surface of the body of the user. Other forms of body attachment to the body may be used, in addition to or instead of adhesive.

Sensor 104 is adapted to be at least partially inserted into the body of the user, where it can make fluid contact with that user's body fluid (e.g., interstitial fluid (ISF), dermal fluid, or blood) and be used, along with the in vivo analyte monitoring circuitry, to measure analyte-related data of the user. Generally, sensor control device 102 and its components can be applied to the body with a mechanical applicator 150 in one or more steps, as described in the incorporated '225 Publication, or in any other desired manner.

After activation, sensor control device 102 can wirelessly communicate the collected analyte data (such as, for example, data corresponding to monitored analyte level and/or monitored temperature data, and/or stored historical analyte related data) to reader device 120 where, in certain embodiments, it can be algorithmically processed into data representative of the analyte level of the user and then displayed to the user and/or otherwise incorporated into a diabetes monitoring regime.

Reader device 120 includes a display 122 that outputs information to the user and/or to accept an input from the user (e.g., if configured as a touch screen), and one or more optional user interface components 121, such as a button, actuator, touch sensitive switch, capacitive switch, pressure sensitive switch, jog wheel or the like. Reader device 120 can also include one or more data communication ports 123 for wired data communication with external devices such as computer system 170. Reader device 120 may also include an integrated or attachable in vitro meter, including an in vitro test strip port (not shown) to receive an in vitro analyte test strip for performing in vitro blood analyte measurements.

Computer system 170 may be a personal or laptop computer, a tablet, or other suitable data processing device. Computer 170 can be either local (e.g., accessible via a direct wired connection such as USB) or remote to reader device 120 and can be (or include) software for data management and analysis and communication with the components in analyte monitoring system 100. Operation and use of computer 170 is further described in the'225 Publication incorporated herein by reference. Analyte monitoring system 100 can also be configured to operate with a data processing module (not shown), also as described in the incorporated '225 Publication.

Trusted computer system 180 can be used to perform authentication of sensor control device 102 and/or reader device 120, used to store confidential data received from devices 102 and/or 120, used to output confidential data to devices 102 and/or 120, or otherwise. Trusted computer system 180 can include one or more computers, servers, networks, databases, and the like. Trusted computer system 180 can be within the possession of the manufacturer or distributor of sensor control device 102, either physically or virtually through a secured connection, or can be maintained and operated by a different party (e.g., a third party). Trusted computer system 180 can be trusted in the sense that system 100 can assume that computer system 180 provides authentic data or information. Trusted computer system 180 can be trusted simply by virtue of it being within the possession or control of the manufacturer, e.g., like a typical web server. Alternatively, trusted computer system 180 can be implemented in a more secure fashion such as by requiring additional password, encryption, firewall, or other internet access security enhancements that further guard against counterfeiter attacks or attacks by computer hackers.

The processing of data and the execution of software within system 100 can be performed by one or more processors of reader device 120, computer system 170, and/or sensor control device 102. For example, raw data measured by sensor 104 can be algorithmically processed into a value that represents the analyte level and that is readily suitable for display to the user, and this can occur in sensor control device 102, reader device 120, or computer system 170. This and any other information derived from the raw data can be displayed in any of the manners described above (with respect to display 122) on any display residing on any of sensor control device 102, reader device 120, or computer system 170. The information may be utilized by the user to determine any necessary corrective actions to ensure the analyte level remains within an acceptable and/or clinically safe range.

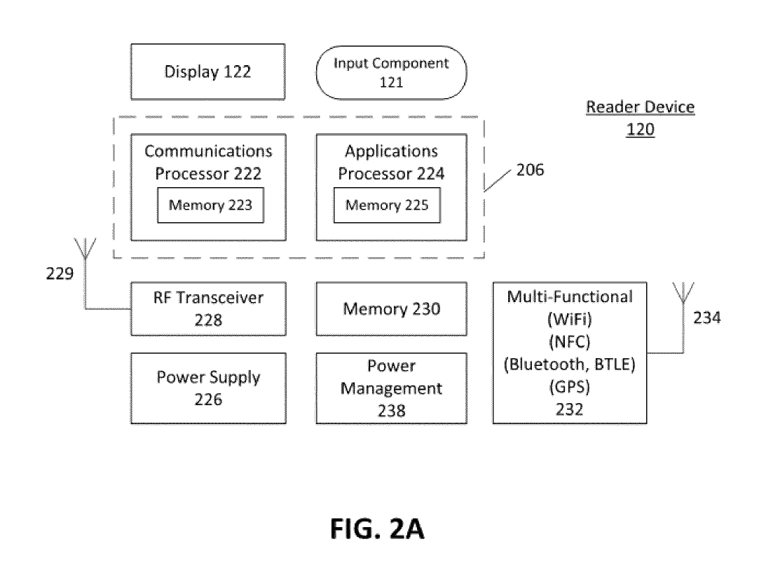

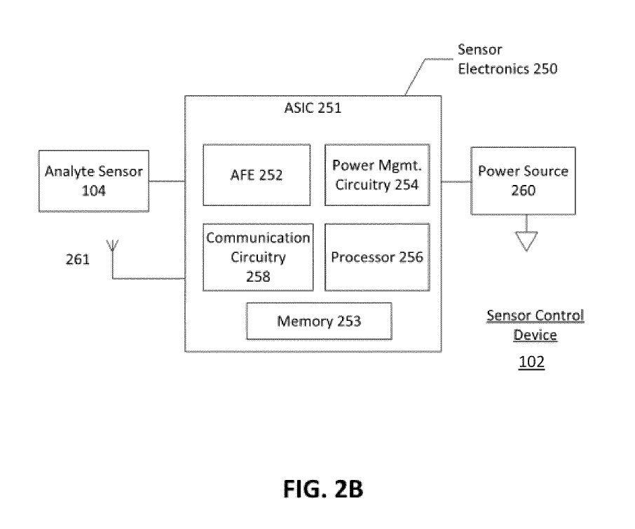

FIGS. 2A-2B depict example embodiments of reader device 120 and sensor control device 102, respectively. As discussed above, reader device 120 can be a mobile communication device such as, for example, a Wi-Fi or internet enabled smartphone, tablet, or personal digital assistant (PDA). Examples of smartphones can include, but are not limited to, those phones based on a WINDOWS operating system, ANDROID operating system, IPHONE operating system, PALM WEBOS, BLACKBERRY operating system, or SYMBIAN operating system, with network connectivity for data communication over the internet or a local area network (LAN).

Reader device 120 can also be configured as a mobile smart wearable electronics assembly, such as an optical assembly that is worn over or adjacent to the user's eye (e.g., a smart glass or smart glasses, such as GOOGLE GLASSES). This optical assembly can have a transparent display that displays information about the user's analyte level (as described herein) to the user while at the same time allowing the user to see through the display such that the user's overall vision is minimally obstructed. The optical assembly may be capable of wireless communications similar to a smartphone. Other examples of wearable electronics include devices that are worn around or in the proximity of the user's wrist (e.g., a watch, etc.), neck (e.g., a necklace, etc.), head (e.g., a headband, hat, etc.), chest, or the like.

FIG. 2A is a block diagram of an example embodiment of a reader device 120 in the form of a smartphone. Here, reader device 120 includes an input component 121, display 122, and processing hardware 206, which can include one or more processors, microprocessors, controllers, and/or microcontrollers, each of which can be a discrete chip or distributed amongst (and a portion of) a number of different chips. Here, processing hardware 206 includes a communications processor 222 having on-board non-transitory memory 223 and an applications processor 224 having on-board non-transitory memory 225. Reader device 120 further includes an RF transceiver 228 coupled with an RF antenna 229, a memory 230, multi-functional circuitry 232 with one or more associated antennas 234, a power supply 226, and power management circuitry 238. FIG. 2A is an abbreviated representation of the internal components of a smartphone, and other hardware and functionality (e.g., codecs, drivers, glue logic, etc.) can of course be included.

Communications processor 222 can interface with RF transceiver 228 and perform analog-to-digital conversions, encoding and decoding, digital signal processing and other functions that facilitate the conversion of voice, video, and data signals into a format (e.g., in-phase and quadrature) suitable for provision to RF transceiver 228, which can then transmit the signals wirelessly. Communications processor 222 can also interface with RF transceiver 228 to perform the reverse functions necessary to receive a wireless transmission and convert it into digital data, voice, and video.

Applications processor 224 can be adapted to execute the operating system and any software applications that reside on reader device 120 (such as any sensor interface application or analyte monitoring application that includes, e.g., SLL 304), process video and graphics, and perform those other functions not related to the processing of communications transmitted and received over RF antenna 229. Any number of applications can be running on reader device 120 at any one time, and will typically include one or more applications that are related to a diabetes monitoring regime, in addition to the other commonly used applications that are unrelated to such a regime, e.g., email, calendar, weather, etc.

Memory 230 can be shared by one or more the various functional units present within reader device 120, or can be distributed amongst two or more of them (e.g., as separate memories present within different chips). Memory 230 can also be a separate chip of its own. Memory 230 is non-transitory, and can be volatile (e.g., RAM, etc.) and/or non-volatile memory (e.g., ROM, flash memory, F-RAM, etc.).

Multi-functional circuitry 232 can be implemented as one or more chips and/or components, including communication circuitry, that perform other functions such as local wireless communications (e.g., Wi-Fi, Bluetooth, Bluetooth Low Energy) and determining the geographic position of reader device 120 (e.g., global positioning system (GPS) hardware). One or more other antennas 234 are associated with both the functional circuitry 232 as needed.

Power supply 226 can include one or more batteries, which can be rechargeable or single-use disposable batteries. Power management circuitry 238 can regulate battery charging and power supply monitoring, boost power, perform DC conversions, and the like. As mentioned, reader device 120 may also include one or more data communication ports such as USB port (or connector) or RS-232 port (or any other wired communication ports) for data communication with computer system 170, or sensor control device 102, to name a few.

FIG. 2B is a block schematic diagram depicting an example embodiment of sensor control device 102 having analyte sensor 104 and sensor electronics 250 (including analyte monitoring circuitry). Although any number of chips can be used, here the majority of the sensor electronics 250 are incorporated on a single semiconductor chip 251 that can be, e.g., a custom application specific integrated circuit (ASIC). Shown within ASIC 201 are several high-level functional units, including an analog front end (AFE) 252, power management circuitry 254, processor 256, and communication circuitry 258 (which can be implemented as a transmitter, receiver, transceiver, passive circuit, or otherwise according to the communication protocol). In this embodiment, both AFE 252 and processor 256 are used as analyte monitoring circuitry, but in other embodiments either circuit can perform the analyte monitoring function. Processor 256 can include one or more processors, microprocessors, controllers, and/or microcontrollers.

A non-transitory memory 253 is also included within ASIC 251 and can be shared by the various functional units present within ASIC 251, or can be distributed amongst two or more of them. Memory 253 can be volatile and/or non-volatile memory. In this embodiment, ASIC 251 is coupled with power source 260, which can be a coin cell battery, or the like. AFE 252 interfaces with in vivo analyte sensor 104 and receives measurement data therefrom and outputs the data to processor 256 in digital form, which in turn processes the data to arrive at the end-result analyte discrete and trend values, etc. This data can then be provided to communication circuitry 258 for sending, by way of antenna 261, to reader device 120 (not shown) where further processing can be performed by, e.g., the sensor interface application. It should be noted that the functional components of ASIC 251 can also be distributed amongst two or more discrete semiconductor chips.

Performance of the data processing functions within the electronics of the sensor control device 102 provides the flexibility for system 100 to schedule communication from sensor control device 102 to reader device 120, which in turn limits the number of unnecessary communications and can provide further power savings at sensor control device 102.

Information may be communicated from sensor control device 102 to reader device 120 automatically and/or continuously when the analyte information is available, or may not be communicated automatically and/or continuously, but rather stored or logged in a memory of sensor control device 102, e.g., for later output.

Data can be sent from sensor control device 102 to reader device 120 at the initiative of either sensor control device 102 or reader device 120. For example, in many example embodiments sensor control device 102 can communicate data periodically in an unprompted or broadcast-type fashion, such that an eligible reader device 120, if in range and in a listening state, can receive the communicated data (e.g., sensed analyte data). This is at the initiative of sensor control device 102 because reader device 120 does not have to send a request or other transmission that first prompts sensor control device 102 to communicate. Broadcasts can be performed, for example, using an active Wi-Fi, Bluetooth, or BTLE connection. The broadcasts can occur according to a schedule that is programmed within device 102 (e.g., about every 1 minute, about every 5 minutes, about every 10 minutes, or the like). Broadcasts can also occur in a random or pseudorandom fashion, such as whenever sensor control device 102 detects a change in the sensed analyte data. Further, broadcasts can occur in a repeated fashion regardless of whether each broadcast is actually received by a reader device 120.

System 100 can also be configured such that reader device 120 sends a transmission that prompts sensor control device 102 to communicate its data to reader device 120. This is generally referred to as “on-demand” data transfer. An on-demand data transfer can be initiated based on a schedule stored in the memory of reader device 120, or at the behest of the user via a user interface of reader device 120. For example, if the user wants to check his or her analyte level, the user could perform a scan of sensor control device 102 using an NFC, Bluetooth, BTLE, or Wi-Fi connection. Data exchange can be accomplished using broadcasts only, on-demand transfers only, or any combination thereof.

Accordingly, once a sensor control device 102 is placed on the body so that at least a portion of sensor 104 is in contact with the bodily fluid and electrically coupled to the electronics within device 102, sensor derived analyte information may be communicated in on-demand or unprompted (broadcast) fashion from the sensor control device 102 to a reader device 120. On-demand transfer can occur by first powering on reader device 120 (or it may be continually powered) and executing a software algorithm stored in and accessed from a memory of reader device 120 to generate one or more requests, commands, control signals, or data packets to send to sensor control device 102. The software algorithm executed under, for example, the control of processing hardware 206 of reader device 120 may include routines to detect the position of the sensor control device 102 relative to reader device 120 to initiate the transmission of the generated request command, control signal and/or data packet.

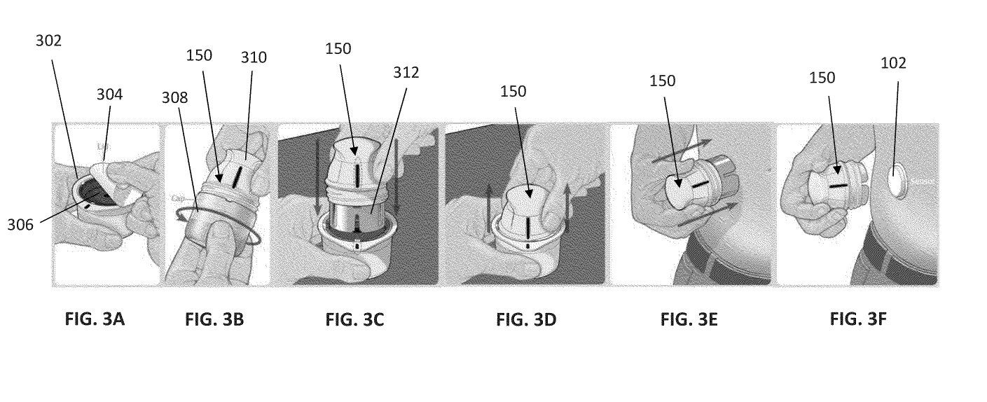

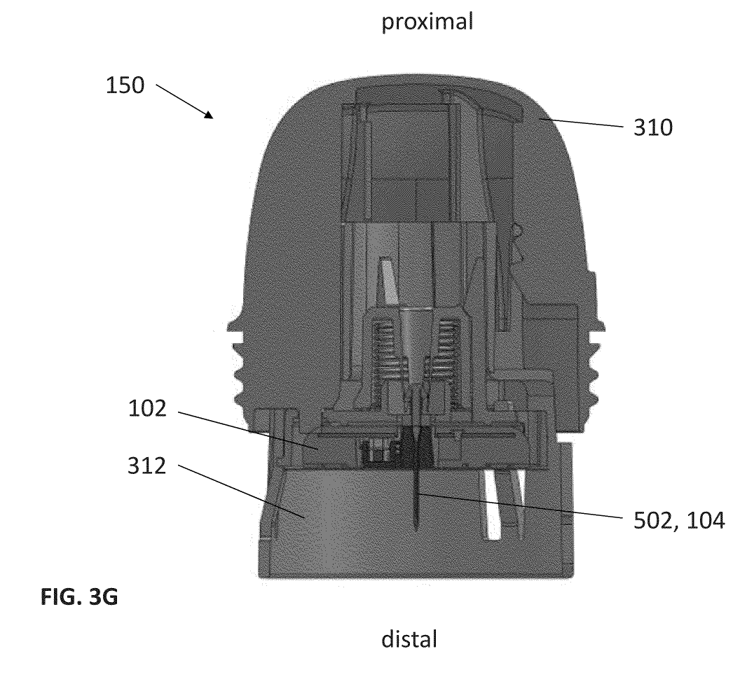

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready device 102 for delivery. FIGS. 3E-3F depict an example embodiment of delivery of sensor control device 102 to an appropriate user location by selecting the appropriate delivery location and applying device 102 to that location.

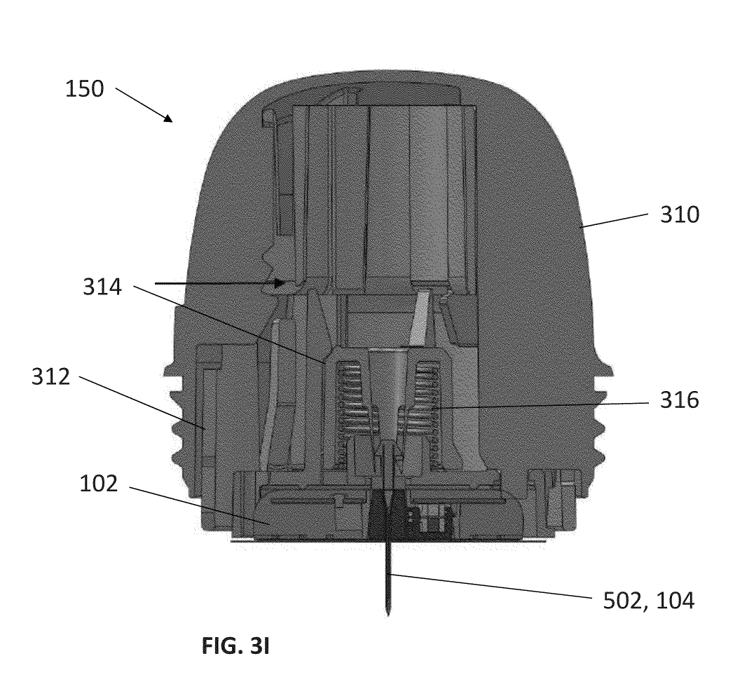

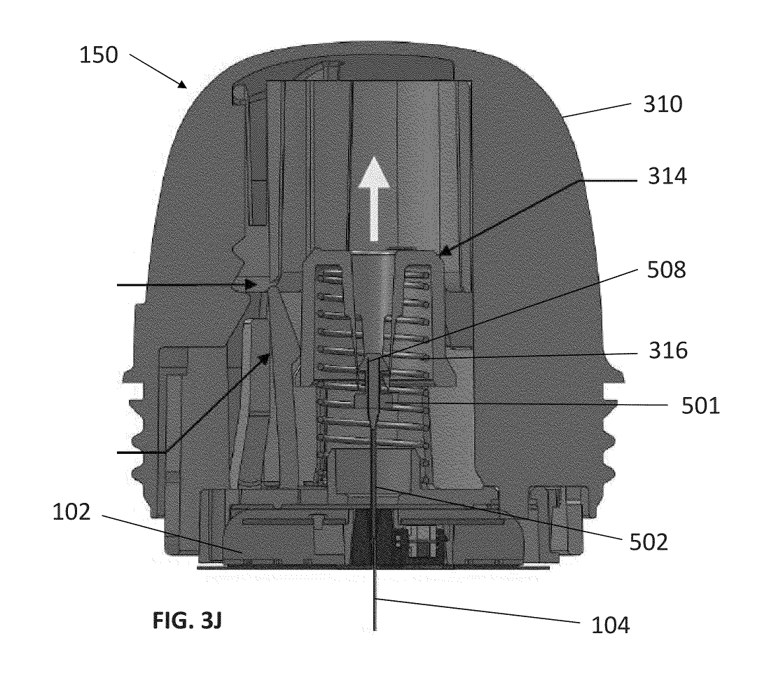

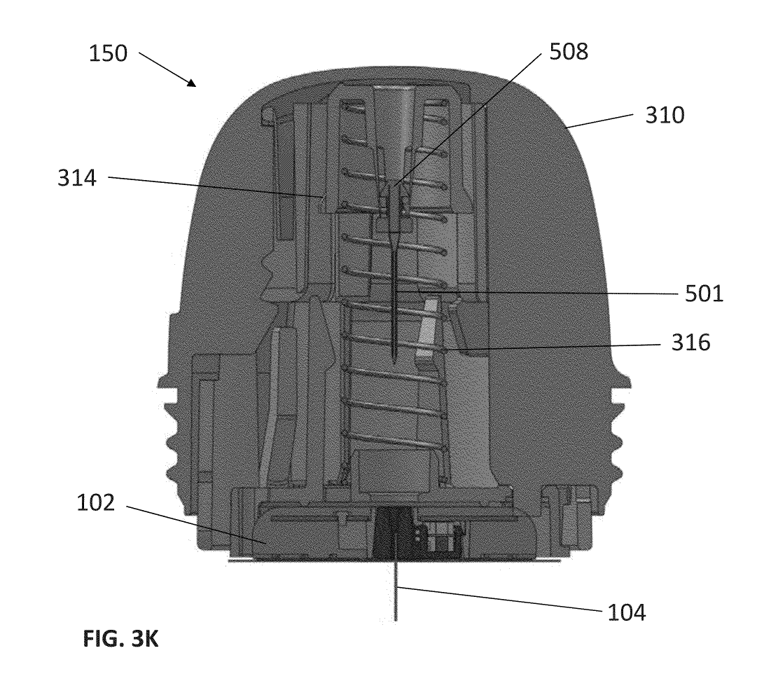

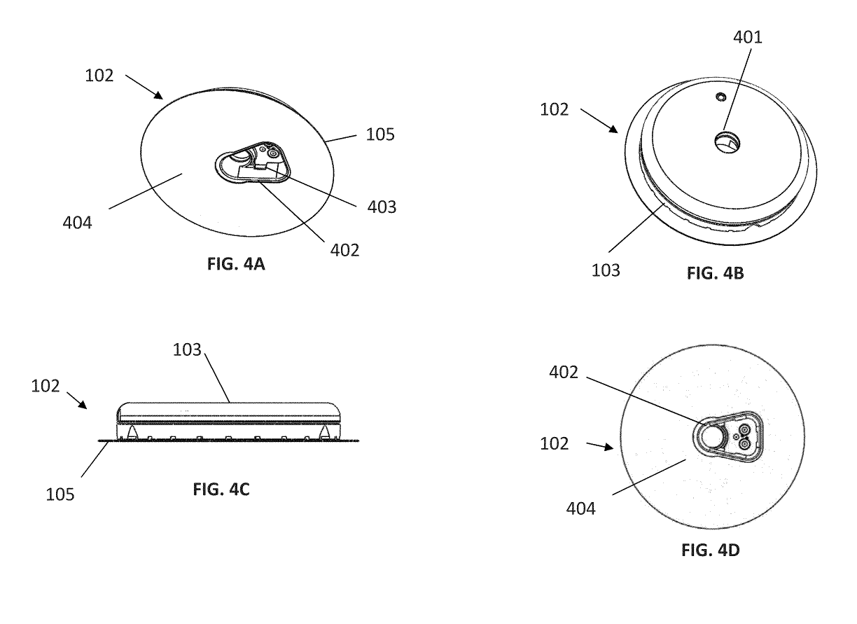

FIG. 3A is a perspective view depicting an example embodiment of a user preparing a container 302, configured here as a tray (although other packages can be used), for an assembly process. The user can accomplish this preparation by removing a lid 304 from tray 302 to expose platform 306. Tray 302 houses a sharp and sensor module 504 (see FIG. 5A) that includes the sharp for puncturing the user's skin and sensor 104 for in vivo measurement of the user's analyte levels.

FIG. 3B is a perspective view depicting an example embodiment of a user preparing an applicator device 150 for assembly. Applicator device 150 can be provided in a sterile package sealed by a cap 308. Preparation of applicator device 150 can include uncoupling (e.g., unscrewing) housing 310 from cap 308 to expose sheath 312 (FIG. 3C). Sensor control device 102 (not shown) is mounted within applicator device 150.

FIG. 3C is a perspective view depicting an example embodiment of a user inserting an applicator device 150 into a tray 302 in order to couple sharp and sensor module 504 with sensor control device 102, for example, to fully assemble device 102. Advancement of sheath 312 against platform 306 unlocks sheath 312 relative to housing 310 and also causes module 504 (not shown) to couple with sensor control device 102 (not shown) within housing 310.

FIG. 3D is a perspective view depicting an example embodiment of a user removing an applicator device 150 from a tray 810 after assembly. The applicator device 150 is removed with sensor control device 102 (not shown) fully assembled with sharp and sensor module 504 and ready for delivery.

FIG. 3E is a perspective view depicting an example embodiment of the sensor control device 102 delivery and insertion process. A user can apply sensor control device 102 using applicator device 150 to a target area of skin, for instance on an abdomen or other appropriate location. Advancing housing 310 towards the skin collapses sheath 312 into housing 310, inserts the sharp and sensor (not shown) into the skin, and applies sensor control device 102 such that the adhesive layer 105 on the bottom side of device 102 adheres to the skin. The sharp is automatically refracted when housing 310 is fully advanced, while the sensor 104 (not shown) is left in position to measure analyte levels. FIG. 3F is a perspective view depicting an example embodiment of a patient with sensor control device 102 in an applied position.

The delivery and insertion operation of applicator device 150 described with respect to FIGS. 3D and 3F is shown and described in greater with respect to FIGS. 3G-K, which are side cross-sectional views. FIG. 3G depicts applicator 150 in a state ready to be positioned against a user's skin. Sensor control device 102 is positioned within housing 310 and/or sheath 312 with sensor 104 and sharp 502 projecting therefrom.

In FIG. 3H, housing 310 has been advanced with respect to sheath 312 but sharp 502 and sensor 104 have not yet exited applicator 150 (e.g., advanced beyond the distal end of sheath 312. In FIG. 3I, housing 310 has been fully advanced by the user's manual push force, and sharp 502 and sensor 104 are extending their maximum distance from the distal end of sheath 312. At this position, a sharp hub carrier 314 is released from a locked position, and a compressed spring 316 is free to push carrier 314 proximally (i.e., away from the skin).

In FIG. 3J, spring 316 has pushed carrier 314 proximally. Carrier 314 is coupled or latched to a groove distal to a proximal end of sharp module 501, in what is referred to as a sharp hub 508. As carrier 314 moves proximally it pulls sharp hub 508 and retracts sharp module 501 from sensor control device 102. In FIG. 3K, spring 316 has expanded a maximum distance and carrier 314 has fully removed sharp module 501 from sensor control device 102, which is adhesively coupled to the user's skin with sensor 104 positioned in vivo. Applicator device 150 can then be removed by the user.

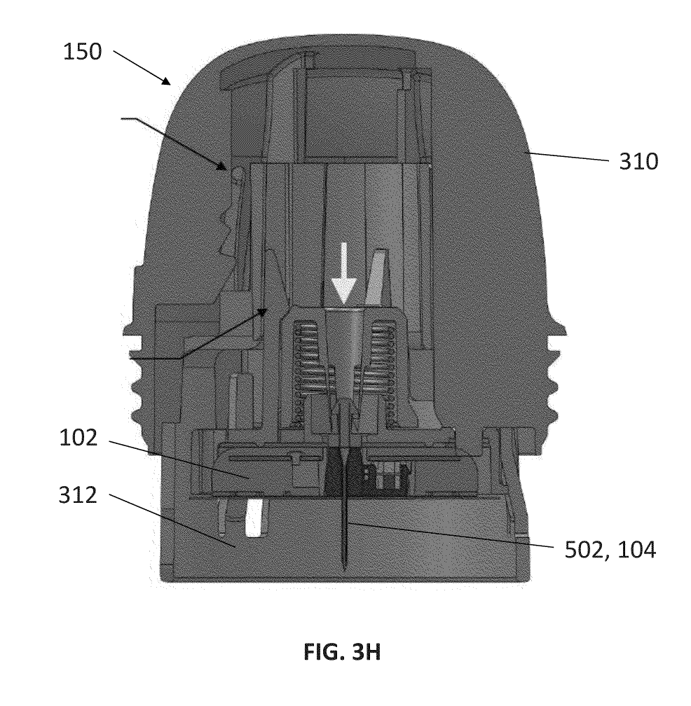

FIGS. 4A-D are distal perspective, proximal perspective, side, and distal end views, respectively, depicting an example embodiment of sensor control device 102 prior to assembly with sharp and sensor module 504. Here, a receptacle 402 exists for receipt of sensor module 504 (not shown) during assembly (such as the assembly process described with respect to FIGS. 3A-D). A channel, lumen, or aperture 401 is present through housing 103 and permits the advancement (during assembly) and retraction (after insertion) of sharp module 501 (not shown) and sharp 502 (not shown). Adhesive 404 is present on adhesive layer 105. Housing 103 can protect sensor electronics 250 (not shown) contained therein and can seal the interior of the device, e.g., for sterility purposes.

FIG. 5A is a perspective view depicting an example embodiment of sharp and sensor module 504 in the state in which it is housed within tray 302 and subsequently assembled into sensor control device 102. Here, module 504 includes a housing 506 and one or more attachment mechanisms 507, such as a deflectable clip, for engaging with a corresponding recess 403 in receptacle 402 of sensor control device 102 (shown in, e.g., FIG. 4A).

Module 504 also includes a sharp module 501, which is also depicted in the perspective view of FIG. 5B. Sharp module 501 includes a sharp hub 508 with a semi-conical or tapered end and a groove positioned between the tapered end and an intermediate outcropping. Sharp hub 508 facilitates engagement with carrier 314 of application device 150 as described and shown with respect to FIGS. 3J-K. Sharp module 501 is slidably removable from a channel in housing 506 of module 504, the channel being aligned with channel 401 of sensor control device 102 (shown in FIG. 4B). Sharp module 501 includes a sharp 502 having a recess or groove 503 in which sensor 104 can be positioned. Sensor 104 and sharp 502 are slidable with respect to each other. As described with respect to FIGS. 3G-K, sharp module 501 can be removed from module 504 after insertion into the user's body. Sensor 104 is left behind, operably coupled with sensor control device 102.

Housing 506 can be made of a relatively rigid plastic material (e.g., polycarbonate and the like), and sharp 502 can be made of a relatively rigid material as well, such as polycarbonate, stainless steel, and the like. During the insertion process, a random or pseudo-random array of forces can be exerted on sharp 502 and sensor 104 by the external environment and the manner in which application device 150 is used. These forces can cause sharp 502 and sensor 104 to vibrate with respect to housing 506 and potentially with respect to each other, and can also cause sharp 502 and sensor 104 to deflect from the position shown in FIG. 5A, which can result in an offset (e.g., non-perpendicular) trajectory into the user's body. These force induced reactions are undesirable as they can potentially disrupt the insertion process, prevent placement of sensor 104 in an ideal location and, to a lesser degree, disrupt the sharp removal process. To reduce the effect of these forces, an elastic dampening mechanism 510 can be placed between sharp 502 and housing 506. This dampening mechanism can absorb the forces applied to sharp 502 and thereby lessen movement of sharp 502 during deployment.

FIGS. 5C-D are perspective views depicting elastic dampening mechanism 510 in greater detail, in a position removed from housing 506. Here, elastic dampening mechanism 510 is configured as an elastic ring-like component. Elastic ring 510 can be positioned within the same channel of module 504 that receives sharp module 501. Elastic ring 510 has a central channel or aperture 511 through which sharp 502 can be advanced and retracted. Also present is a groove 512 and flat face 514 for assisting in orienting ring 510 during assembly and for creating a friction fit with recess 516 in housing 506. Groove 512 interfaces with a complementary shaped abutment (not shown) within housing 506. Similarly, the edges of recess 516 are shaped to be complementary to the flat and curved side faces 514 and 515, respectively, of ring 510.

Dampening mechanism 510 can be coupled with housing 506 in any manner desired, including, but not limited to, the use of a friction fit, adhesive, or with a molding process (e.g., two-shot molding). The fit between mechanism 510, housing 506, and sharp 502 should be relatively tight to provide optimal dampening. Mechanism 510 can be formed from an elastomer that exhibits sufficient dampening characteristics including, but not limited to, thermoplastic elastomers (TPE), fluoroelastomers (such as FKM), silicon rubber, and the like.

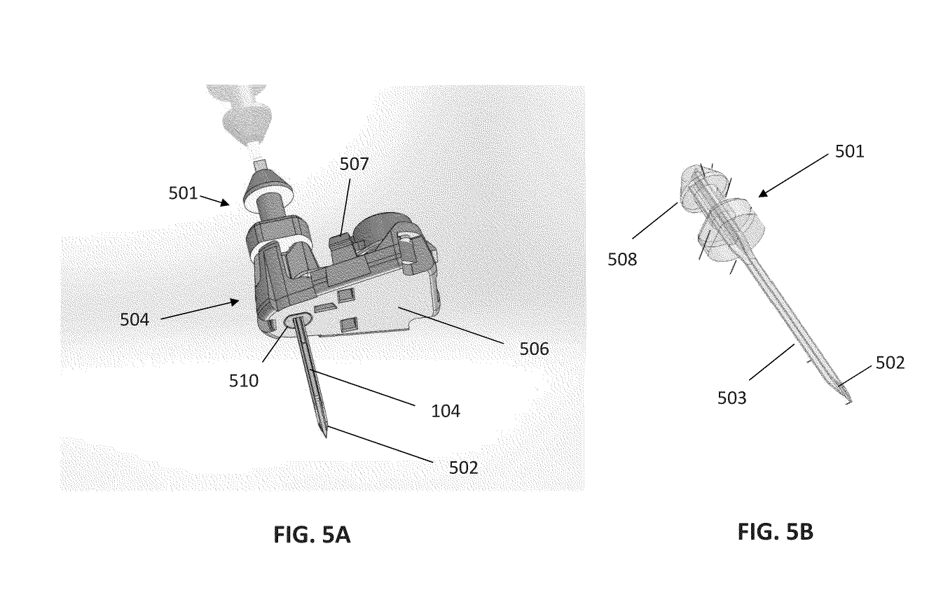

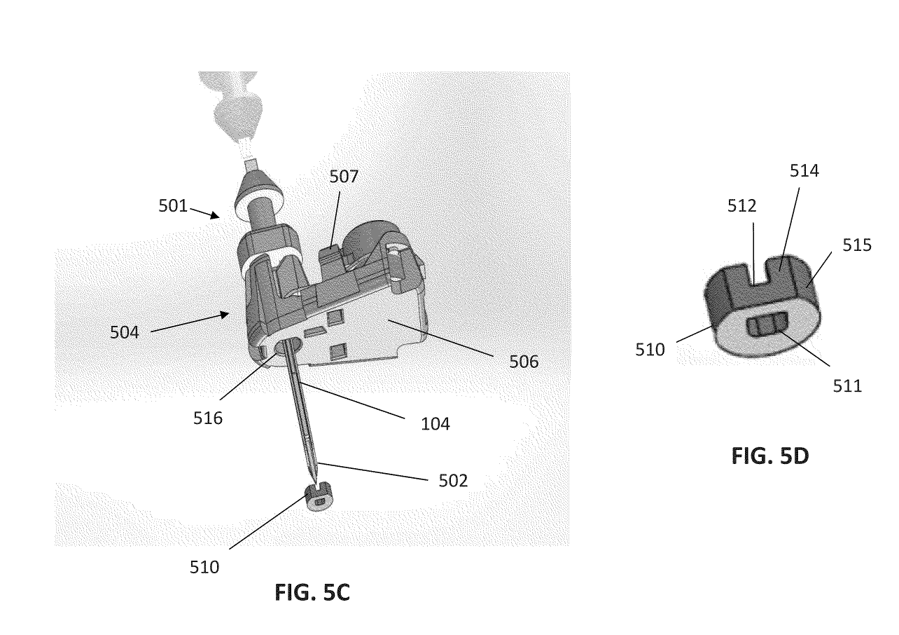

In many embodiments, sensor control device 102 can be sterilized and sealed within its housing 103 such the interior of the device is inaccessible to the external material environment (e.g., air and the user). In such a configuration the user does not have access to power source 260, which in many embodiments is a battery. Out of the factory, sensor electronics 250 can be in a dormant state where only a very low power drain exists on the sealed power source 260. When the user is ready to use a new sensor control device 102 for the first time, the sensor control device 102 can be brought out of its dormant state into a relatively higher power state, or a full power state (e.g., awakened or activated) by a mechanism activated by the user. This enhances both the shelf and operating life of sensor control device 102. Such activation mechanisms are described herein with reference to FIGS. 6A, 6B, and 7.

FIG. 6A is a top down view depicting an example embodiment of a sensor control device 102 (such as that discussed with reference to FIGS. 1 and 2B) configured for use with a switch for bringing sensor control device 102 out of a low power state and into a relatively higher or full power state. In some embodiments, this switch can be a low-cost, user-friendly, electronic switch that can be activated by the user by simply removing an external conductive tab that disconnects an electrical connection between two nodes and activates sensor electronics 250, which in turn controls the operation of sensor 104. The switch can be implemented in hardware or a combination of both hardware and software.

FIG. 6A depicts the top side of sensor housing 103 with adhesive patch 105 on the bottom side of housing 103 visible around the periphery of device 102. On the top surface of sensor housing 103 is a removable conductive element 604. Here, this element 604 is configured as an adhesive tape 604 with an insulating material on a top side and a conductive material 605 on a bottom side (indicated with dashed line as obscured beneath the top side of tape 604). When attached to housing 103, the bottom side conductive material 605 of adhesive tape 604 provides an electrical contact with a first conductive contact 602 (shown with dashed line as obscured beneath tape 604) and a second conductive contact 603 (also obscured). The conductive material of tape 604 provides an electrical connection between both contacts 602 and 603, which represent electrical nodes A and B, respectively. In this state, nodes A and B are shorted together and at the same voltage that is determined by the circuit described with respect to FIG. 7.

The insulated top side of tape 604 isolates the nodes and conductive surface from the surrounding environment. This insulation can also be present on the bottom side of tape 604 in region 606 around the periphery of conductive material 605. In some embodiments, the tape adhesive can be located only in region 606, in which case it can be insulating. In embodiments where the adhesive is present over the conductive material 605 then such adhesive can be conductive to provide for improved electrical contact. In some embodiments, a conductive adhesive is present only in the regions of tape 604 directly over contacts 602 and 603. In other embodiments, a conductive adhesive is present over the entire conductive material 605 surface but not in region 606. In still other embodiments, adhesive is present across the entire bottom side of tape 604. Furthermore, while conductive material 605 is shown to present in a generally rectangular area, and contacts 602 and 603 are shown as generally circular, other profile shapes can be used.

Adhesive is absent from one side or end of tape 604 that forms a pull tab 608 by which the user can grasp tape 604 and pull to remove it from the top surface of sensor control device 102. While depicted here as being located on the top surface of housing 103, tape 604 and contacts 602 and 603 can also be present on the side of housing 103 or on the bottom of housing 103 (in which case tape 604 is removed prior to deploying device 102 on the user's skin) In some embodiments, tape 604 is removed after delivery of sensor control device 102 to the body (e.g., after the step depicted in FIG. 3F). In other embodiments, tape 604 is removed prior to delivery of sensor control device 102 to the body (either before or after assembly). In these embodiments, the user has access to tape 604 through application device 150 if needed.

FIG. 6B depicts another embodiment of sensor control device 102 where conductive material 605 is present only in the general areas of contacts 602 and 603. The electrical connection between contacts 602 and 603 is formed by a wire, strip, or trace of conductive material 609 present on the underside of tape 604. Material 609 can have an insulating jacket if desired. Instead of being placed on the underside, wire 609 can be embedded within the insulating material of tape 604. Wire 609 can be soldered or adhered to the patches of conductive material 605.

FIG. 7 is a circuit schematic depicting an example embodiment of switch 601. Here, switch 601 includes a power source 704 and an RC network that includes a capacitor 706 and a resistor 708. Power source 704 can include an anode terminal and a cathode terminal. The circuitry of switch 601 can be present, for example, in the power management circuitry 254 of ASIC 251 (see FIG. 2B). Power source 704 can be the same single power source 260 used to operate sensor control device 102, or power source 704 can be a stand-alone separate and one-time use battery present within device 102 in addition to power source 260.

FIG. 7 depicts the conductive nodes A and B between which the removable tape 604 is disposed. Switch 601 further includes a ground as shown and a voltage output to digital logic 710 of sensor electronics 250.

The removable adhesive conducting tape 604 is disposed between nodes A and B, and removable therefrom. When implemented with the circuitry depicted in FIG. 7, the removable tape 604 can be a user-removable “pull-tab” providing a switch that enables the user to initiate electrical power to digital logic 710 of sensor electronics 250.

Power source 704 charges capacitor 706 to the same voltage across the electrical connection provided by tape 604. Removal of tape 604 breaks the connection between power source 704 and capacitor 706 and starts an RC discharge of capacitor 706. Node B follows the exponential discharge curve to reach a lower voltage after a time t. Digital logic 710 (e.g., processor 256) connected to the switch 601 circuitry can detect the logic 1 to logic 0 (i.e., a reverse binary logic) transition that serves as a signal for activation. Once activated, processor 256 can save the activated state change into memory and can be programmed or otherwise configured to ignore any future transitions on node B, thus making switch 601 suitable only for one time use, e.g., a “one-time switch.” If power source 704 is the same single power source 260 used to operate sensor control device 102, then one or more additional connections to sensor electronics 250 are present from node A to enable source 704 to supply power after connection 604 is removed.

Switch 601 can also be considered a reverse binary switch. When node B is connected to node A, the switch is not enabled, but when the tape 604 is removed and the capacitor 706 is allowed to discharge to zero (or the reference potential), the digital logic switch no longer registers the power received from power source 704. Tape 604 can be located external to the sensor control device 102 and made accessible to the user thereby without user access to internal components of the sensor housing 103.

The resistance (R) of the resistor 708 can be chosen to be relatively large to minimize drain on power source 704 during storage. For example, in one embodiment, R=20 Mega ohms (Mohms) with a 3 volt source 704, the current drain is 0.15 uA, which depletes source 704 by 6.6 milliamp hours (mAhr). If the source capacity is 250 mAhr, the source capacity will be depleted by about 2.6% after 5 years in storage.

Referring still to FIG. 7, choosing the capacitance (C) of capacitor 706 to be relatively small allows a timely discharge to activate sensor electronics 250. This also facilitates meeting size and cost constraints that are oftentimes considered in selecting capacitor 706. For example, when C=0.1 microfarads and R=20 Mohms, it will take about 4 seconds for the RC discharge to reach 0.4 volts, which can be recognized as a logic 0 voltage level. This is the delay between the time that the electrical connection between nodes A and B is broken by removal of tape 604 to the time sensor electronics 250 detects the logic 0 value.

While these embodiments have been described with respect to a removable conductive tape, other mechanisms or elements for breaking the connection between nodes A and B can be used. Also, a secondary switch can be included such that activation of switch 601 can notify digital logic 710 that electronics 250 should be awakened, at which point a second switch can be tripped to either create a full connection between source 704 (e.g., source 260) and electronics 250 or to create a connection between an alternate primary power source 260 (e.g., other than source 704) and electronics 250.

Turning now to the chemical aspects of system 100, analytes that may be monitored with system 100 include, but are not limited to, acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

Analyte sensor 104 may include an analyte-responsive enzyme to provide a sensing element. Some analytes, such as oxygen, can be directly electrooxidized or electroreduced on sensor 104, and more specifically at least on a working electrode (not shown) of a sensor 104. Other analytes, such as glucose and lactate, require the presence of at least one electron transfer agent and/or at least one catalyst to facilitate the electrooxidation or electroreduction of the analyte. Catalysts may also be used for those analytes, such as oxygen, that can be directly electrooxidized or electroreduced on the working electrode. For these analytes, each working electrode includes a sensing element proximate to or on a surface of a working electrode. In many embodiments, a sensing element is formed near or on only a small portion of at least a working electrode.

Each sensing element includes one or more components constructed to facilitate the electrochemical oxidation or reduction of the analyte. The sensing element may include, for example, a catalyst to catalyze a reaction of the analyte and produce a response at the working electrode, an electron transfer agent to transfer electrons between the analyte and the working electrode (or other component), or both.

A variety of different sensing element configurations may be used. In certain embodiments, the sensing elements are deposited on the conductive material of a working electrode. The sensing elements may extend beyond the conductive material of the working electrode. In some cases, the sensing elements may also extend over other electrodes, e.g., over the counter electrode and/or reference electrode (or counter/reference where provided). In other embodiments, the sensing elements are contained on the working electrode, such that the sensing elements do not extend beyond the conductive material of the working electrode. In some embodiments a working electrode is configured to include a plurality of spatially distinct sensing elements. Additional information related to the use of spatially distinct sensing elements can be found in U.S. Provisional Application No. 61/421,371, entitled “Analyte Sensors with Reduced Sensitivity Variation,” which was filed on Dec. 9, 2010, and which is incorporated by reference herein in its entirety and for all purposes.

The terms “working electrode”, “counter electrode”, “reference electrode” and “counter/reference electrode” are used herein to refer to conductive sensor components, including, e.g., conductive traces, which are configured to function as a working electrode, counter electrode, reference electrode or a counter/reference electrode respectively. For example, a working electrode includes that portion of a conductive material, e.g., a conductive trace, which functions as a working electrode as described herein, e.g., that portion of a conductive material which is exposed to an environment containing the analyte or analytes to be measured, and which, in some cases, has been modified with one or more sensing elements as described herein. Similarly, a reference electrode includes that portion of a conductive material, e.g., conductive trace, which function as a reference electrode as described herein, e.g., that portion of a conductive material which is exposed to an environment containing the analyte or anlaytes to be measured, and which, in some cases, includes a secondary conductive layer, e.g., a Ag/AgCl layer. A counter electrode includes that portion of a conductive material, e.g., conductive trace which is configured to function as a counter electrode as described herein, e.g., that portion of a conductive trace which is exposed to an environment containing the analyte or anlaytes to be measured. As noted above, in some embodiments, a portion of a conductive material, e.g., conductive trace, may function as either or both of a counter electrode and a reference electrode. In addition, “working electrodes”, “counter electrodes”, “reference electrodes” and “counter/reference electrodes” may include portions, e.g., conductive traces, electrical contacts, or areas or portions thereof, which do not include sensing elements but which are used to electrically connect the electrodes to other electrical components.

Sensing elements that are in direct contact with the working electrode, e.g., the working electrode trace, may contain an electron transfer agent to transfer electrons directly or indirectly between the analyte and the working electrode, and/or a catalyst to facilitate a reaction of the analyte. For example, a glucose, lactate, or oxygen electrode may be formed having sensing elements which contain a catalyst, including glucose oxidase, glucose dehydrogenase, lactate oxidase, or laccase, respectively, and an electron transfer agent that facilitates the electrooxidation of the glucose, lactate, or oxygen, respectively.

In other embodiments the sensing elements are not deposited directly on the working electrode, e.g., the working electrode trace. Instead, the sensing elements may be spaced apart from the working electrode trace, and separated from the working electrode trace, e.g., by a separation layer. A separation layer may include one or more membranes or films or a physical distance. In addition to separating the working electrode trace from the sensing elements, the separation layer may also act as a mass transport limiting layer and/or an interferent eliminating layer and/or a biocompatible layer.

In certain embodiments which include more than one working electrode, one or more of the working electrodes may not have corresponding sensing elements, or may have sensing elements that do not contain one or more components (e.g., an electron transfer agent and/or catalyst) needed to electrolyze the analyte. Thus, the signal at this working electrode may correspond to background signal which may be removed from the analyte signal obtained from one or more other working electrodes that are associated with fully-functional sensing elements by, for example, subtracting the signal.

In certain embodiments, the sensing elements include one or more electron transfer agents. Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes including ferrocene. Examples of inorganic redox species are hexacyanoferrate (III), ruthenium hexamine, etc. Additional examples include those described in U.S. Pat. Nos. 6,736,957, 7,501,053 and 7,754,093, the disclosures of each of which are incorporated herein by reference in their entirety.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used, without a polymeric component.

Embodiments of polymeric electron transfer agents may contain a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation.

Another example of an ionically-bound mediator is a positively charged polymer including quaternized poly (4-vinyl pyridine) or poly(1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, including 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(1-vinyl imidazole).

Embodiments may employ electron transfer agents having a redox potential ranging from about −200 mV to about +200 mV versus the standard calomel electrode (SCE). The sensing elements may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, including a glucose oxidase, glucose dehydrogenase (e.g., pyrroloquinoline quinone (PQQ), dependent glucose dehydrogenase, flavine adenine dinucleotide (FAD) dependent glucose dehydrogenase, or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent, which, as described above, may be polymeric. A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

In certain embodiments, the sensor works at a low oxidizing potential, e.g., a potential of about +40 mV vs. Ag/AgCl. These sensing elements use, for example, an osmium (Os)-based mediator constructed for low potential operation. Accordingly, in certain embodiments the sensing elements are redox active components that include: (1) osmium-based mediator molecules that include (bidente) ligands, and (2) glucose oxidase enzyme molecules. These two constituents are combined together in the sensing elements of the sensor.

A mass transport limiting layer (not shown), e.g., an analyte flux modulating layer, may be included with the sensor to act as a diffusion-limiting barrier to reduce the rate of mass transport of the analyte, for example, glucose or lactate, into the region around the working electrodes. The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may provide many functions, e.g., biocompatibility and/or interferent-eliminating functions, etc. A mass transport limiting layer may be applied to an analyte sensor as described herein via any of a variety of suitable methods, including, e.g., dip coating and slot die coating.

In certain embodiments, a mass transport limiting layer is a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

A membrane may be formed by crosslinking in situ a polymer, modified with a zwitterionic moiety, a non-pyridine copolymer component, and optionally another moiety that is either hydrophilic or hydrophobic, and/or has other desirable properties, in an alcohol-buffer solution. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly (ethylene glycol), hydroxyl or polyhydroxyl modifiers, may be used to enhance the biocompatibility of the polymer or the resulting membrane.

A membrane may be formed in situ by applying an alcohol-buffer solution of a crosslinker and a modified polymer over the enzyme-containing sensing elements and allowing the solution to cure for about one to two days or other appropriate time period. The crosslinker-polymer solution may be applied over the sensing elements by placing a droplet or droplets of the membrane solution on the sensor, by dipping the sensor into the membrane solution, by spraying the membrane solution on the sensor, and the like. Generally, the thickness of the membrane is controlled by the concentration of the membrane solution, by the number of droplets of the membrane solution applied, by the number of times the sensor is dipped in the membrane solution, by the volume of membrane solution sprayed on the sensor, or by any combination of these factors. In order to coat the distal and side edges of the sensor, the membrane material may have to be applied subsequent to singulation of the sensor precursors. In some embodiments, the analyte sensor is dip-coated following singulation to apply one or more membranes. Alternatively, the analyte sensor could be slot-die coated wherein each side of the analyte sensor is coated separately. A membrane applied in the above manner may have any combination of the following functions: (1) mass transport limitation, i.e., reduction of the flux of analyte that can reach the sensing elements, (2) biocompatibility enhancement, or (3) interferent reduction.

In some embodiments, a membrane composition for use as a mass transport limiting layer may include one or more leveling agents, e.g., polydimethylsiloxane (PDMS). Additional information with respect to the use of leveling agents can be found, for example, in US Patent Application Publication No. US 2010/0081905, the disclosure of which is incorporated by reference herein in its entirety.

In some instances, the membrane may form one or more bonds with the sensing elements. The term “bonds” is intended to cover any type of an interaction between atoms or molecules that allows chemical compounds to form associations with each other, such as, but not limited to, covalent bonds, ionic bonds, dipole-dipole interactions, hydrogen bonds, London dispersion forces, and the like. For example, in situ polymerization of the membrane can form crosslinks between the polymers of the membrane and the polymers in the sensing elements. In certain embodiments, crosslinking of the membrane to the sensing element facilitates a reduction in the occurrence of delamination of the membrane from the sensor.

According to several embodiments, analyte sensor 104 is factory calibrated and manufactured with minimal sensor-to-sensor variation. Calibration parameters from the factory calibration can be stored in the sensor control device 102 to allow for algorithmic correction to the measured analyte data by device 102 or device 120. Sensor 104 can be worn for up to 14 days, without the need for any user calibration. This feature differs from other existing sensors which require multiple fingerstick capillary blood glucose (BG) measurements for calibration.

Capillary and venous BG measurements are typically used as reference to evaluate the accuracy of these and other sensor devices. Venous samples analyzed using a laboratory analyzer, such as the YSI, have been used by clinical laboratories for the calibration of sensors, whereas the users use a capillary BG measurement for sensor calibration. Glucose concentration between the capillary and venous samples may differ due to differences in blood sample composition and the rate of consumption of glucose in the tissues, and therefore the sample type used for calibration versus reference measurement may influence the results of an accuracy evaluation.

The use of capillary BG as a comparator to in vivo fluid (e.g., dermal, interstitial) sensor readings from the analyte sensor presents an appropriate primary end point in evaluating the performance and accuracy of this factory calibrated glucose monitoring system 100. Embodiments of the systems, devices, and methods described herein can be exemplified in or practiced by (or with) the FreeStyle Libre Flash Glucose Monitoring System (developed by Abbott Diabetes Care Inc., Alameda, Calif.) has been designed to address some of the unmet needs of glucose monitoring. Several features distinguish this sensor from existing sensor technology. The wired enzyme sensor 104 is factory calibrated requiring no other user calibration during 14 days of wear, and is disposable after use. A dedicated hand held reader 120 with built-in blood glucose meter is used to scan the sensor to receive up to 8 hours of interstitial glucose readings. An embodiment of system 100, as exemplified by the FreeStyle Libre System, displays trends and alerts on reader 120, but does not have alarms, which may provide a good option for individuals who are overwhelmed by alarms or complain of alarm fatigue.

In a first study, the participants inserted the sensor on the back of each upper arm for up to 14 days. Three sensor lots were used, and each was factory calibrated. There were 3 scheduled in-clinic visits of at least 8 hours during the 14-day sensor wear period. At least 8 capillary blood glucose (BG) tests, using the FreeStyle Precision blood glucose meter built into the reader, which were performed daily. Tests were completed whether at home or during in-clinic visits. The preferred testing was upon waking, before each meal, an hour after each meal and at bedtime. After each BG test, participants obtained a sensor reading. Sensor readings were masked to participants who were asked to maintain their established diabetes management plan. Capillary BG tests coincided with venous samples (YSI reference) drawn during in-clinic visits.

A mixed model was used to assess sensitivity and mean absolute relative difference (MARD) as these parameters depend on subjects wearing the sensor (random effect) and factors such as sensor lot and insertion site (fixed effect). The mixed model analysis accounts for both random and fixed effects. Analyses were carried out using SAS version 9.2 (SAS Institute, Cary, N.C.).

Seventy-two (72) of 75 study participants were included in the evaluation. Three participants exited after Visit 1 (2 did not respond or could not comply with study visits, and 1 had non-study related severe hypoglycemia prior to sensor insertion with unknown complications).

The mean age (±SD) of the study participants was 46±15 years (range 18 to 71 years). The mean weight was 182.2 pounds±42.1 (range 102 to 300 pounds), and the mean Body Mass Index (BMI) was 28.3±5.3 (range 18.7 to 47.2). The mean time since diagnosis of diabetes was 23.0±13.1 years (range 2.4 to 50.6 years). Of the 72 participants that completed the study, 50% (36/72) were men and 90.3% (65/72) were White. The majority of participants 81.9% (59/72) had type 1 diabetes, and 54.2% (39/72) used an insulin pump. The majority of study participants complied with the preferred BG test schedule with an average >7.7 tests per day of sensor wear.

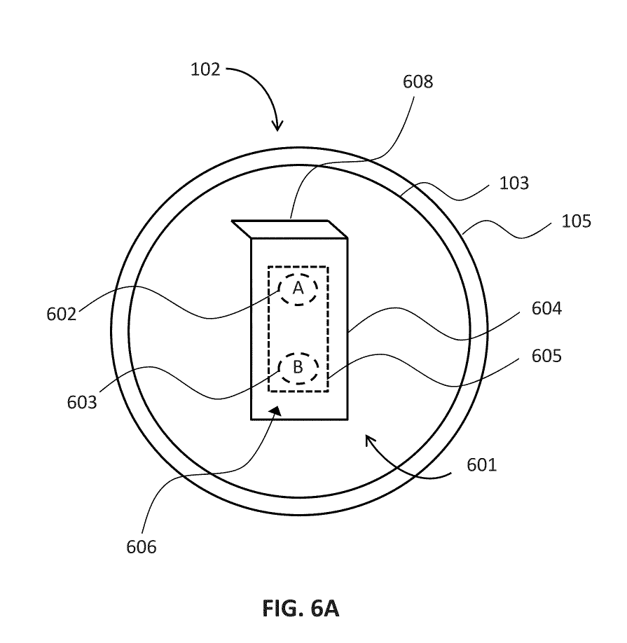

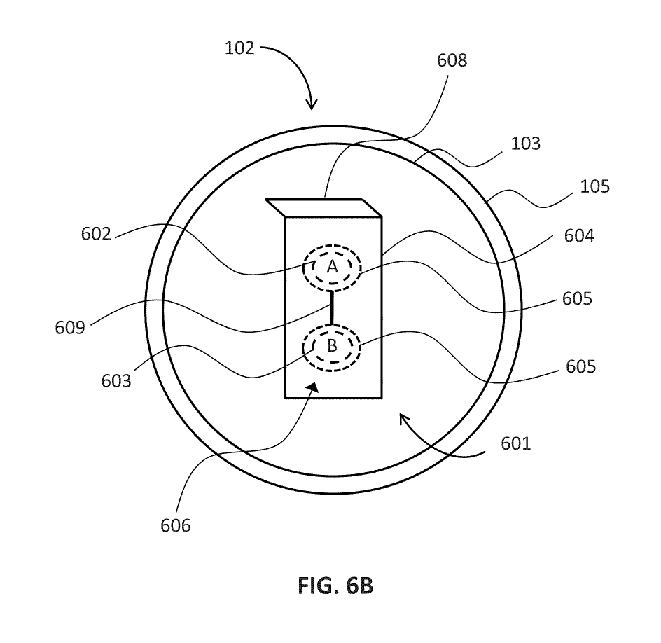

A typical sensor profile of this first test study is shown in FIG. 8. Sensor profiles illustrated sensor performance throughout the 14-day wear period, including day time and night time wear. Data automatically stored by the sensor every 15 minutes, current sensor glucose values shown on the reader, and capillary BG reference measurements are represented in the profile. A total of 13195 BG and 12172 YSI reference results were paired with sensor glucose results.

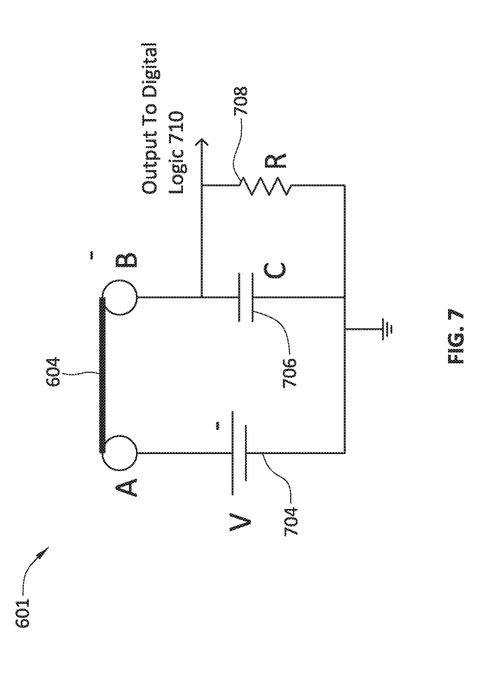

System 100 demonstrated accuracy, with 86.7% of sensor results within Zone A of the Consensus Error Grid with a BG reference (FIG. 9). The percentage of real-time sensor results in Zones A and B of the Consensus Error Grid was 99.7%. The percentage of sensor results in Zone A of the Consensus Error Grid was 83.1%, 87.4% and 89.8% for each individual Sensor lot. Sensor results in Zone A and B of the Consensus Error Grid were similar among the three sensor lots (99.8%, 99.5% and 99.8%).

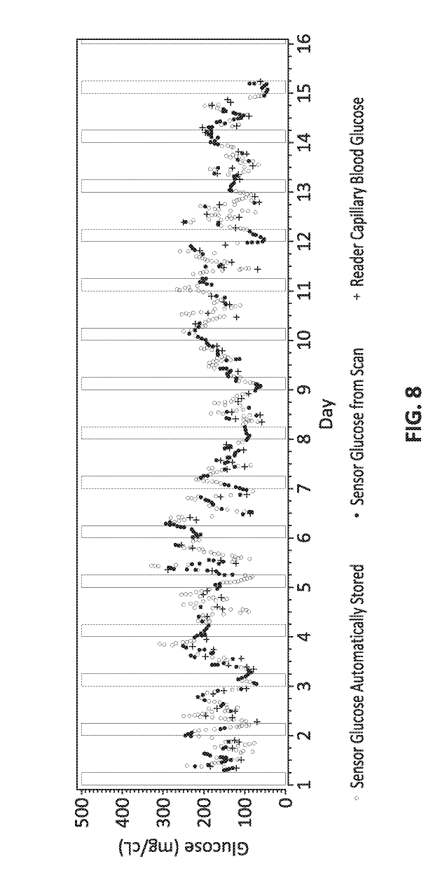

The overall MARD was 11.4% for sensor results with capillary BG reference. MARD results for each of the three sensor lots under evaluation were 11.8%, 11.6% and 10.8% using BG reference. The variation in MARD for different sensors is shown in FIG. 10, with the mixed model estimating the between subjects variance component to be 44% of the total variation. There was no significant difference between left and right arm insertion sites (11.2% and 11.4%, p=0.595).

The FreeStyle Libre sensor results were highly correlated to capillary BG. Regression analysis produced a slope of 1.02, an intercept of −6.4 mg/dL, and a correlation coefficient of 0.95 for the FreeStyle Libre sensor using capillary BG reference (with range 23 mg/dL to 498 mg/dL). The sensitivity, as measured by slope, coefficient of variation between sensors was 8.5%, 8.6% and 8.9% for each sensor lot. This variation was predominantly due to between sensor variance with the mixed model estimating the between subjects variance component to be 37% of the total. There were no statistically significant difference in sensor sensitivity (ie, slope) between insertion sites on either the right or left arm (p=0.554).

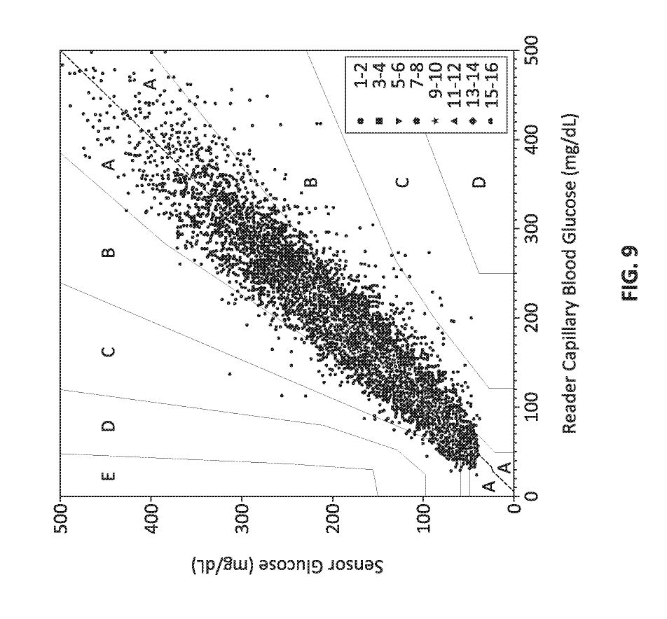

Performance was stable across the 14 days of wear. The percentage of readings within Consensus Zone A (BG reference) on Days 2, 7, and 14 was 88.4%, 89.2%, and 85.2%, respectively (FIG. 11).

The mean time to first glucose results was 1 hour and 1 minute (n=168), and 100% of sensors were able to provide interstitial glucose results within 1 hour and 10 minutes after insertion. The mean lag time between the FreeStyle Libre sensor and YSI reference was 4.5±4.8 minutes.

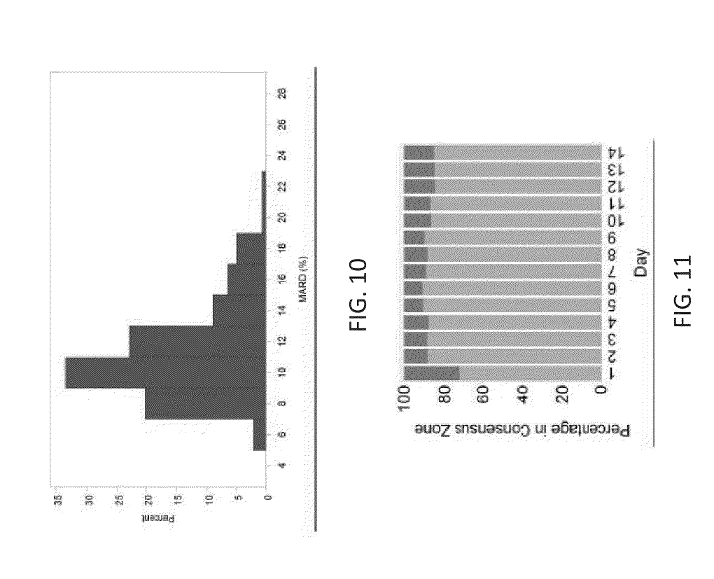

Sensor accuracy was not affected by factors such as BMI, age, type of diabetes, clinical site, insulin administration, or HbA1C, as the percentage of readings within the Consensus Zone A was similar (FIG. 12).

In a study of 55 subjects who wore a wired enzyme, factory calibrated sensor for up to 14 days, results demonstrated MARD of 12.2%, with 88.0% of sensor readings within Zone A of Consensus EGA, and minimal change in sensor sensitivity over the 14-day wear time.

Similarly, another study evaluating the feasibility of a factory calibrated sensor using wired enzyme technology for 5 days demonstrated MARD of 13.4%, and 83.5% of readings were within Zone A of Consensus EGA. In comparison, the study reported that multiple fingerstick calibrations resulted in MARD of 12.7% and 84.1% of readings within Zone A. Results from those published studies were consistent with the accuracy and sensor duration outcomes in the present study. Accuracy of system 100 was similar across 14 days of sensor wear, with the exception of the 1st day of wear which had the lowest accuracy (Consensus EGA showed 72.0% in Zone A for day 1 compared to 88.8% in Zone A for day 2). This may be due in part to the body's natural inflammatory response to sensor insertion, which has been shown to affect glucose concentration in interstitial fluid.

In the present study, the FreeStyle Libre sensor did not show any marked differences in accuracy outcomes relative to BMI, age, type of diabetes, clinical site, insulin administration or HbA1C. The present study with the FreeStyle Libre System, included a broad range of BMI (18.7 kg/m2 to 47.2 kg/m2) which did not affect the sensor accuracy. Placement of the FreeStyle Libre sensor was on both arms for each subject and future studies could evaluate the effects of sensor accuracy in different locations on the body.

A second single-arm US clinical study was conducted with seventy-two (72) study participants with type 1 or type 2 diabetes enrolled at four (4) clinical sites.

Study participants inserted and wore the sensor on the back of each upper arm (two sensors total) for up to 14 days. Three factory-only calibrated production sensor lots were used for this second study. This number is consistent with the industry practice to demonstrate the performance of reagent system across multiple production lots. There were three scheduled in-clinic visits during the 14-day sensor wear period, where venous blood samples were collected every 15 minutes over 8 hours for YSI reference. The first in-clinic visit was between Day 1 and Day 3, second in-clinic visit was between Day 4 and Day 9, and the third in-clinic visit was between Day 10 and Day 14. At least eight (8) capillary BG measurements, using the BG meter built into the reader, were required to be performed on each day of the sensor wear. One BG test strip lot was used to minimize lot to lot variation. Tests were completed whether at home or during in-clinic visits. The preferred testing was upon waking, before each meal, an hour after each meal and at bedtime. Immediately after each BG measurement, participants obtained a confirmation of a successful sensor scan. Sensor readings were masked to participants who were asked to maintain their established diabetes management plan. There was no manipulation of the glucose levels of the subjects except for their normal meal and insulin doses. Capillary BG measurements coincided with venous YSI samples drawn during in-clinic visits.

A linear mixed model was used to assess sensitivity and MARD between insertion sites, with subject as a random effect and insertion site (left arm, right arm) and lot as fixed effects. The lag between the sensor 104 and YSI reference was evaluated using a model that characterizes delay with a time constant. Analyses were carried out using SAS version 9.2 (SAS Institute, Cary, N.C.).

Seventy-two (72) of 75 study participants were included in the evaluation. Three participants exited after Visit 1 (2 could not comply with study visits, and 1 had non-study related severe hypoglycemia prior to sensor insertion with unknown complications). The baseline subject characteristics of the study participants are provided in Table 1 shown below.

TABLE 1

| Characteristic | | Mean ± SD | Median | Range |
|---|---|---|---|---|
| Age (years) | | 46.4 ± 15.1 | 48.5 | 18 to 71 |
| Weight | pounds | 182.2 ± 42.1 | 175.8 | 102.0 to 299.8 |
| | kilograms | 82.6 ± 19.1 | 79.7 | 46.3 to 136.0 |
| Height | inches | 67.1 ± 4.3 | 66.5 | 59 to 81 |
| | meters | 1.70 ± 0.11 | 1.69 | 1.50 to 2.06 |

| | | | |
|---|---|---|---|
| Body Mass Index | 28.3 ± 5.3 | 27.4 | 18.7 to 47.2 |
| Years since diagnosis | 23.0 ± 13.1 | 22.3 | 2.4 to 50.6 |
| Total number of injections per | 4.6 ± 1.1 | 4 | 3 to 8 |
| day (N subjects = 33) | | | |
| HbA1c (%) | 7.8 ± 1.2 | 7.8 | 5.5 to 11.5 |
| Number of BG tests per day | 7.4 ± 1.0 | 7.7 | 4.0 to 9.6 |

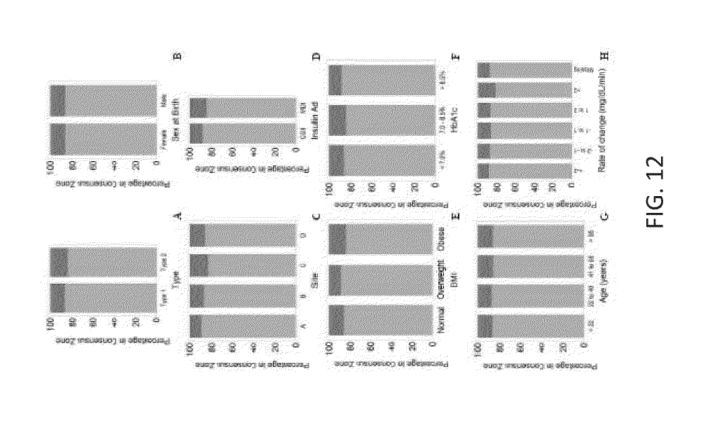

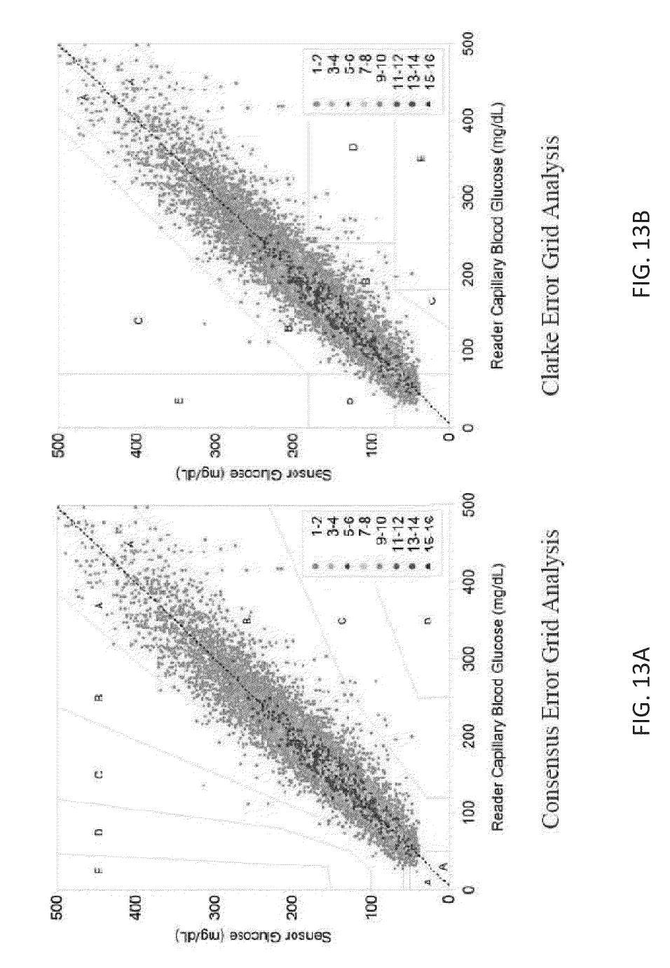

Real-time glucose values were available for 99.2% (25834/26045) of sensor scans, where complete sensor data was transferred to the reader. A total of 13195 BG measurements and 12172 YSI reference results were paired with sensor glucose results. Twenty eight pairs were excluded because the reference glucose result was beyond the BG system dynamic range (20-500 mg/dL), and 114 pairs were excluded because the sensor result was beyond system 100's dynamic range (40-500 mg/dL). The percentage of results in the Zone A of the Consensus and Clarke Error Grid was 86.7% and 85.5%, respectively, as shown in FIGS. 13A and 13B. The percentage of sensor results in Zones A and B of the Consensus and Clarke Error Grid was 99.7% and 99.0%, respectively while 86.2% and 82.8% of sensor results were within ±15 mg/dL or ±20% of BG reference and venous reference, respectively. Continuous Glucose Error Grid Analysis (CG-EGA) versus venous reference showed 96.5% (11232/11640) of the data is categorized as clinically accurate, and a further 2.4% (274/11640) as benign errors.

The overall MARD was 11.4% for sensor results with capillary BG measurements. The overall MARD in the clinic alone for sensors results with capillary BG measurements and with YSI reference was 12.1% and 12%, respectively. A detailed difference analysis against BG capillary measurements and venous blood reference is provided in Table 2 below.

TABLE 2

| | | | | | | | | | | | Without Hypo/ | |
|---|---|---|---|---|---|---|---|---|---|---|---|---|
| | Glucose | | Days 1-14 | | | Days 2-14 | | | Day | Night | rapid change^{a} | |
| Reference | Range | Parameter | All | Home | Clinic | All | Home | Clinic | 7 AM-11 PM | 11 PM-7 AM | All | Clinic |
| Capillary, | <100, mg/dL | MAR, | 11.3 | 11.3 | 11.8 | 11.0 | 11.0 | 10.6 | 11.5 | 10.4 | 10.3 | 8.4 |
| BG | | mg/dL | | | | | | | | | | |
| | | N | 2153 | 1946 | 207 | 1985 | 1813 | 172 | 1787 | 366 | 905 | 97 |
| | ≧100 mg/dL | MARD, % | 10.7 | 10.5 | 11.8 | 10.2 | 10.2 | 10.4 | 10.8 | 9.9 | 10.2 | 11.1 |
| | | N | 11042 | 9642 | 1400 | 9987 | 8878 | 1109 | 9717 | 1325 | 9341 | 1236 |
| | All | All, % | 11.4 | 11.3 | 12.1 | 11.0 | 11.0 | 10.7 | 11.5 | 10.8 | 10.4 | 11.0 |
| | | N | 13195 | 11588 | 1607 | 11972 | 10691 | 1281 | 11504 | 1691 | 10246 | 1333 |
| Venous, | <100, mg/dL | MAR, | | | 13.4 | | | 12.6 | | | | 10.2 |
| YSI | | mg/dL | | | | | | | | | | |
| | | N | | | 1475 | | | 1327 | | | | 620 |
| | ≧100 mg/dL | MARD, % | | | 11.4 | | | 10.3 | | | | 10.9 |
| | | N | | | 10697 | | | 8789 | | | | 9722 |
| | All | All, % | | | 12.0 | | | 11.0 | | | | 11.0 |
| | | N | | | 12172 | | | 10116 | | | | 10342 |

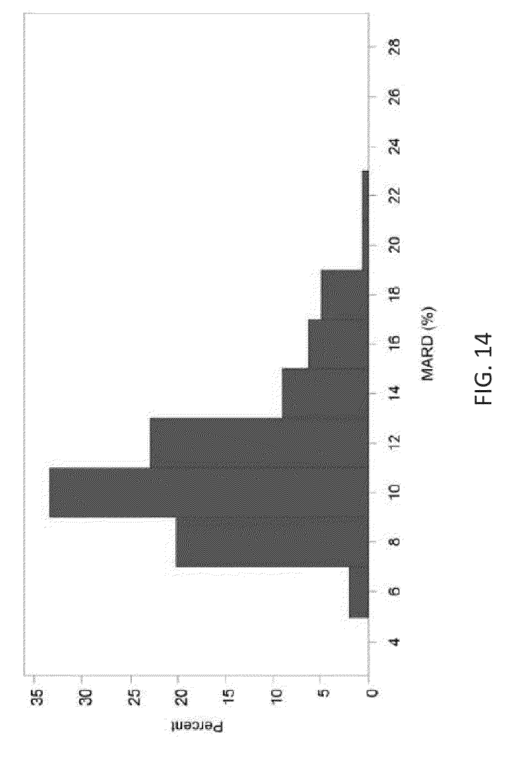

The variation in MARD against BG measurements for different sensors is shown in FIG. 14, with the linear mixed model estimating the between subjects variance component to be 44% of the total variation. There was no significant difference between left and right arm insertion sites (11.2% and 11.4%, p=0.5950).

System 100's sensor results were highly correlated to capillary BG measurements. Regression analysis produced a slope of 1.02, an intercept of −6.4 mg/dL, and a correlation coefficient of 0.95 (with range 23 mg/dL to 498 mg/dL). The coefficient of variation of sensitivity (as measured by slope) between sensors was 8.5%, 8.6% and 8.9% for each sensor lot. This variation was predominantly due to between sensor variance with the linear mixed model estimating the between subjects variance component to be 37% of the total. There were no statistically significant difference in sensor sensitivity (i.e., slope) between insertion sites on either the right or left arm (p=0.5542).

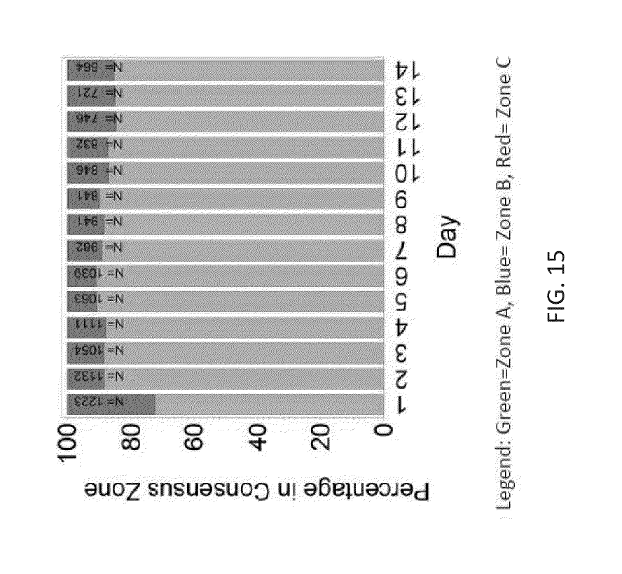

Performance of system 100 was stable across the 14 days of wear after the first day. The percentage of readings within Consensus Zone A (BG measurements) on Day 2, Day 7, and Day 14 was 88.4%, 89.2%, and 85.2%, respectively as shown in FIG. 15, and the MARD on the same days was 11.9%, 10.9% and 10.8% respectively. The mean time to first glucose results was 1 hour and 1 minute (n=168), and 100% of sensors were able to provide interstitial glucose results within 1 hour and 10 minutes after insertion. The mean lag time between sensor 104 and YSI reference was 4.5±4.8 minutes.

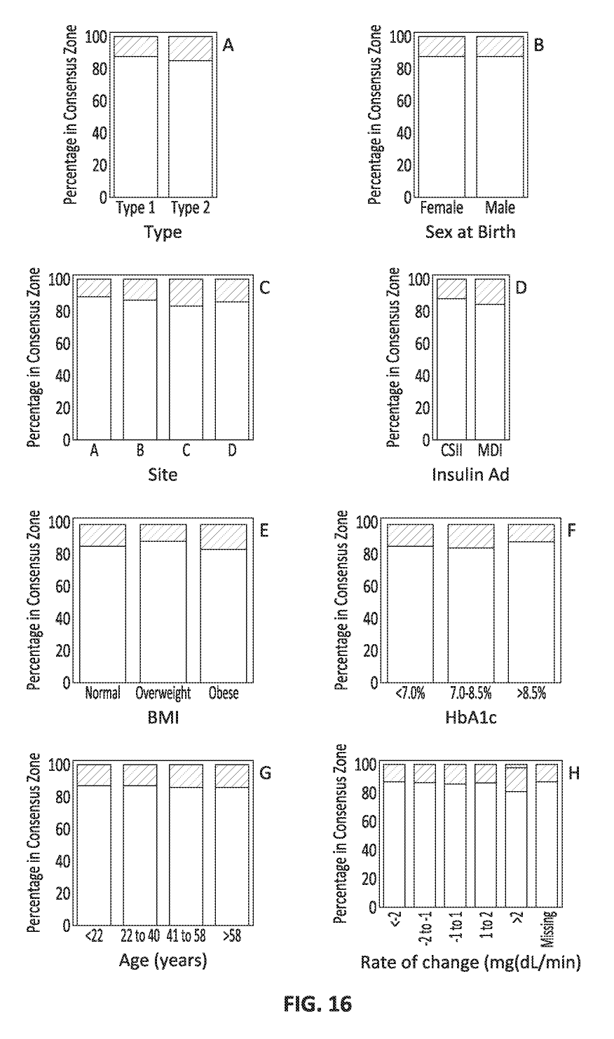

Sensor accuracy was not affected by factors such as BMI, age, type of diabetes, clinical site, insulin administration, or HbA1C, as the percentage of readings within the Consensus Zone A was similar. This is shown in FIG. 16 with the following Legend: Green=Zone A, Blue=Zone B, Red=Zone C. Abbreviation Key: BMI=Body Mass Index; Insulin Ad=Insulin Administration; HbA1c=Hemoglobin A1c.

This study evaluated the performance and usability of the flash glucose monitoring system. Study results showed agreement between system 100's sensor readings and capillary BG measurements as well as venous reference. The capillary BG reference provided a wider distribution of glucose results and covered up to 14 days of wear. Therefore, capillary BG measurement was used as the primary comparator for system 100's performance evaluation. Capillary BG measurement provides more reference points and represents real life accuracy during daily patient use.

System 100 has a benefit in that the wired enzyme factory-only calibrated sensor has sensor wear time of multiple days or weeks (e.g., 14 days) without additional calibration. This lack of reliance on an external BG monitor for calibration is a potential advantage as errors in capillary BG meters could potentially lead to system errors. In vivo sensors requiring routine user calibration several times daily can be affected by glucose instability, such as observed post-prandially. Delays or lag between interstitial readings and venous or capillary readings have also been shown to vary among sensors, with newer generation sensors demonstrating less lag time.

Differences between interstitial, capillary, and venous readings are also considered when comparing accuracy outcomes. Sources contributing to differences between capillary BG measurement versus venous YSI readings include the amount of blood used for testing, delays in analysis from the time of sampling, and differences in the composition of the blood samples.

Collectively, these differences limit the direct comparison of accuracy outcomes among sensor technologies. Therefore, the present study was compared with reported outcomes with similar wire enzyme technology, factory calibrated sensors, and those reporting accuracy outcomes using Consensus Error Grid Analysis (EGA). In a study of 55 subjects who wore a wired enzyme, factory calibrated sensor for up to 14 days, results demonstrated MARD of 12.2%, with 88.0% of sensor readings within Zone A of Consensus EGA, and minimal change in sensor sensitivity over the 14-day wear time. Similarly, another study evaluating the feasibility of a factory calibrated sensor using wired enzyme technology for 5 days demonstrated MARD of 13.4%, and 83.5% of readings were within Zone A of Consensus EGA. Results from these published studies were consistent with the outcomes in the present study. Accuracy of the System 100 was similar across 14 days of sensor wear, with the exception of the 1^{st} day of wear which had the lowest accuracy (Consensus EGA showed 72.0% in Zone A for day 1 compared to 88.4% in Zone A for day 2). This may be due in part to the body's natural inflammatory response to sensor insertion, which has been shown to affect glucose concentration in interstitial fluid.

In the present study, sensor 104 did not show any marked differences in accuracy outcomes relative to BMI, age, type of diabetes, clinical site, insulin administration or HbA1C. In comparison, the accuracy (Clarke EGA) of the FreeStyle Navigator® sensor, as reported by Weinstein et al, did not differ as a function of age, sex, ethnicity, years since diagnosis of diabetes, or sensors worn on either the arm or abdomen but differed depending on the subject's BMI. The percentage of readings in Zone A (Clarke EGA) for participants who had BMI of <25 kg/m² was 78.8% compared to 84.4% for participants with BMI >30 kg/m², which the authors suggested could have been attributed to differences in blood flow relative to subcutaneous adipose tissue thickness. The present study with the FreeStyle Libre System included a broad range of BMI (18.7 kg/m² to 47.2 kg/m²) which did not affect the sensor accuracy. Placement of sensor 104 was on both arms for each subject, and future studies could evaluate the effects of sensor accuracy in different locations on the body.

These results have clinical implications for individuals with diabetes and for the clinicians who treat them. Several randomized controlled studies have revealed better HbA1C outcomes associated with the frequency of sensor wear. Thus, an in vivo sensor with a longer wear period that does not require fingerstick calibration with its associated burden and pain, may support more frequent sensor use with improved glycemic outcomes. This system 100 with no additional fingerstick tests may also benefit groups that have demonstrated poor adoption of persistent sensor use.

Sensor control device 102 provides a broader interval and volume of measurements, including day and night readings, which can be used to evaluate glucose patterns and trends. In comparison, capillary BG measurements provide single, intermittent measurements, which may not capture intervals of extreme variability or nocturnal events. In a recent study, it was demonstrated that the use of continuous glucose monitoring with or without alarms reduces time spent outside glucose targets compared with in vitro blood glucose measurements with BG meters. System 100 can provide the user with the current, real time glucose result, glucose pattern and trend information on the display of the handheld reader when the sensor is scanned. This type of monitoring system 100 may benefit individuals who have ceased sensor use due to alarm fatigue, becoming overwhelmed by alarms.

In this prospective study, the performance of the factory-only calibrated flash glucose monitoring system 100 was demonstrated by the accuracy of sensor readings and the stability of accurate readings over 14 days of use. The accuracy of system 100 was unaffected by subject characteristics, making it suitable for a broad range of individuals. Under normal conditions, system 100 can provide an easy to use and comfortable sensor wear experience for up to 14 days without the need for fingerstick measurements. It is anticipated that the provision of comprehensive glucose data for up to 14 days with reduced pain and burden for the end user will support enhanced diabetes management.

These results have clinical implications for individuals with diabetes and for the clinicians who treat them. Accuracy has been demonstrated for a factory calibrated sensor with wear duration of up to 14 days. Moreover, the sensor-to-sensor results demonstrated stable accuracy and minimal variation across the factory calibrated sensors. Several randomized controlled studies have revealed better HbA1C outcomes associated with the frequency of sensor wear. Thus, a sensor with a longer wear period that does not require additional fingerstick calibration with its associated burden and pain, may support more frequent sensor use with improved glycemic outcomes.

The FreeStyle Libre sensor provides a broader interval and volume of measurements, including day and night readings, which can be used to evaluate glucose patterns and trends. In comparison, capillary BG readings provide single, intermittent measurements, which may not capture intervals of extreme variability or nocturnal events. The FreeStyle Libre System provides the user with the current glucose result, glucose pattern and trend information on the display of the handheld reader when the sensor is scanned. This type of monitoring system may benefit individuals who have ceased sensor use due to alarm fatigue or those who become overwhelmed by alarms as well as individuals who have experienced the difference between capillary blood glucose readings and interstitial glucose readings with some CGM systems.

An analyte monitoring system 100 in accordance with one embodiment of the present disclosure comprises an analyte sensor 104 in fluid contact with bodily fluid (e.g., interstitial or dermal) under a skin surface to generate signals corresponding to a monitored analyte level in the bodily fluid, sensor electronics 250 electrically coupled to analyte sensor 104 to process the signals generated by analyte sensor 104 and to communicate the processed signals generated by analyte sensor 104, and a data receiver 120 in communication with sensor electronics 250 to receive the processed signals from sensor electronics 250, wherein the received processed signals correspond to a monitored analyte level in the fluid having a mean absolute relative difference of 12% or less.

In certain embodiments, the received processed signals corresponding to the monitored analyte level in interstitial fluid have a mean absolute relative difference of 11.4%. In certain embodiments, the received processed signals corresponding to the monitored analyte level in the interstitial fluid in Zones A and B of the Consensus Error Grid is 99.8%.

In certain embodiments, the received processed signals corresponding to the monitored analyte level in the interstitial fluid in Zone A of the Consensus Error Grid is 89.8%.

An analyte monitoring device in accordance with one embodiment includes an analyte sensor in fluid contact with interstitial fluid under a skin surface to generate signals corresponding to a monitored analyte level in the interstitial fluid, and sensor electronics electrically coupled to the analyte sensor to process the signals generated by the analyte sensor, wherein the processed signals corresponding to the monitored analyte level in the interstitial fluid have a mean absolute relative difference of 12% or less.

In certain embodiments, the processed signals corresponding to the monitored analyte level in the interstitial fluid have a mean absolute relative difference of 11.4%. In certain embodiments, the processed signals corresponding to the monitored analyte level in the interstitial fluid in Zones A and B of the Consensus Error Grid is 99.8%. In certain embodiments, the processed signals corresponding to the monitored analyte level in the interstitial fluid in Zone A of the Consensus Error Grid is 89.8%.

In certain embodiments, the analyte sensor includes a glucose sensor having a plurality of electrodes, where the plurality of electrodes include a working electrode comprising an analyte-responsive enzyme and/or a mediator. In certain embodiments, at least one of the analyte-responsive enzyme and the mediator is chemically bonded to a polymer disposed on the working electrode. In certain embodiments, the at least one of the analyte-responsive enzyme and the mediator is crosslinked with the polymer.

Various other modifications and alterations in the structure and method of operation of this disclosure will be apparent to those skilled in the art without departing from the scope and spirit of the embodiments of the present disclosure. Although the present disclosure has been described in connection with particular embodiments, it should be understood that the present disclosure as claimed should not be unduly limited to such particular embodiments. It is intended that the following claims define the scope of the present disclosure and that structures and methods within the scope of these claims and their equivalents be covered thereby.

For each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories, power sources, communication circuits, transmitters, receivers, processors and/or controllers that can be programmed to execute any and all method steps or facilitate the execution of any and all method steps. These sensor control device embodiments can be used and can be capable of use to implement those steps performed by a sensor control device from any and all of the methods described herein. Likewise, embodiments of reader devices are disclosed having one or more transmitters, receivers, memories, power sources, processors and/or controllers that can be programmed to execute any and all method steps or facilitate the execution of any and all method steps. These embodiments of the reader devices can be used to implement those steps performed by a reader device from any and all of the methods described herein. Embodiments of trusted computer systems are also disclosed. These trusted computer systems can include one or more processors, controllers, transmitters, receivers, memories, databases, servers, and/or networks, and can be discretely located or distributed across multiple geographic locales. These embodiments of the trusted computer systems can be used to implement those steps performed by a trusted computer system from any and all of the methods described herein.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

The subject matter described herein and in the accompanying figures are done so with sufficient detail and clarity to permit the inclusion of claims, at any time, in means-plus-function format pursuant to 35 U.S.C. section 112, part (f). However, a claim is to be interpreted as invoking this means-plus-function format only if the phrase “means for” is explicitly recited in that claim.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A sensor control device, comprising:
a power source comprising a first terminal and a second terminal;
an analyte sensor including a plurality of electrodes, including an in vivo portion of the analyte sensor configured for fluid contact with a bodily fluid under a skin surface, the analyte sensor configured to monitor an analyte level in the bodily fluid and to generate one or more signals associated with the monitored analyte level;
a switch comprising:
a first node electrically coupled to the first terminal;
a second node electrically coupled to a capacitor, the capacitor being electrically coupled
in parallel with a resistor, the capacitor and resistor being electrically coupled to the second terminal; and
a removable conductive element electrically connecting the first node with the second node, wherein removal of the removable conductive element causes discharge of the capacitor; and
sensor electronics configured to control the analyte sensor, the sensor electronics adapted to detect discharge of the capacitor and to transition from a low power state to a relatively high power state after detection of the discharge.

2. The sensor control device of claim 1, wherein the removable conductive element is an adhesive tape.

3. The sensor control device of claim 2, wherein the adhesive tape comprises a conductive material on a bottom surface of the adhesive tape and an insulating material on an opposing upper surface of the adhesive tape, the conductive material of the adhesive tape electrically connecting the first node with the second node.

4. The sensor control device of claim 1, wherein the sensor electronics are connected to the second node and the electronics comprise:
a sensor interface section configured to electrically couple to the plurality of electrodes of the analyte sensor; and
one or more processors programmed to process the one or more signals from the analyte sensor for filtering, calibration, storage, transmission, or one or more combinations thereof, the processor also being programmed to detect discharge of the capacitor and to cause transition from the low power state to the relatively high power state after detection of the discharge.

5. The sensor control device of claim 4, wherein the processor detects discharge of the capacitor in the form of a digital logic 1 to logic 0 transition in voltage from the switch.

6. The sensor control device of claim 1, wherein after transition to the relatively high power state the sensor electronics stores the state change into memory.

7. The sensor control device of claim 6, wherein the sensor electronics are further configured to ignore subsequent transitions of voltage from the second node of the switch.

8. The sensor control device of claim 1, wherein the switch and sensor electronics are configured such that the switch can only be used once to cause a transition in a power state of the sensor control device.

9-58. (canceled)

59. A method of activating a sensor control device, wherein the sensor control device comprises: a power source comprising a first terminal and a second terminal; a switch comprising a first node electrically coupled to the first terminal and a second node electrically coupled to a capacitor, the capacitor being electrically coupled in parallel with a resistor, and the capacitor and resistor being electrically coupled to the second terminal; and sensor electronics configured to control an analyte sensor, wherein the method comprises:
removing a conductive element from the sensor control device such that the first node is electrically disconnected from the second node and discharge of the capacitor commences;
detecting discharge of the capacitor with the sensor electronics; and
transitioning the sensor electronics from a low power state to a relatively high power state.

60. The method of claim 59, further comprising:
coupling the sensor control device with the analyte sensor such that the sensor control device is in an assembled state; and
prior to removing the conductive element, attaching the sensor control device in its assembled state to a body of the user with the analyte sensor positioned in vivo.

61. The method of claim 59, wherein the removable conductive element is an adhesive tape.

62. The method of claim 59, further comprising, after removing the conductive element, then ignoring, by the sensor electronics, any further voltage transition on the second node.
